(19) United States
(12) Patent Application Publication
(10) Pub. No.: US 2025/0134688 A1
(43) Pub. Date: May 1, 2025

(54) Ankle Orthoses for High Ankle Sprain And Related Methods

(71) Applicant: DJO, LLC, Carlsbad, CA (US)

(72) Inventors: David Madden, Forest Lake, MN (US); Jeremy Nathanson, Vista, CA (US); Robert B. Anderson, Charlotte, NC (US); Gregory Charles Berlet, Westerville, OH (US); W. Hodges Davis, Charlotte, NC (US); James Calder, Alresford (GB); Nathan Moore, Memphis, TN (US)

(21) Appl. No.: 18/932,260

(22) Filed: Oct. 30, 2024

Related U.S. Application Data
(60) Provisional application No. 63/546,593, filed on Oct. 31, 2023.

(51) Int. Cl. A61F 5/01

(52) U.S. Cl. CPC A61F 5/0111

(57) ABSTRACT

Ankle orthoses include a fabric portion having closure flaps, a rigid upright assembly having at least a portion disposed within the fabric portion, and at least one adjustable cuff assembly configured to encircle the lower leg of the wearer above the ankle over the fabric portion and apply medial-lateral force to the lower leg of the wearer, thereby, preventing a tibia and fibula of the wearer from separating. The orthosis includes a first fastener coupled to a side of the orthosis at a position inferior of the ankle joint of the wearer; and a first midfoot strap configured to wrap at least partly around the midfoot of the wearer over the fabric portion and having a first end coupled to the same position on the side of the orthosis as the first fastener, and a second end configured to couple to the first fastener. Additional orthoses and methods are provided.

### CROSS-REFERENCE TO RELATED APPLICATIONS

This application claims priority to U.S. Provisional Patent Application No. 63/546,593, filed Oct. 31, 2023, the contents of which are each hereby incorporated by reference in their entirety.

### BACKGROUND

### Field of the Disclosure

The present disclosure relates generally to ankle orthoses for treating both general and high ankle sprains and related methods.

### Description of the Related Technology

Previous ankle braces have employed straps, including figure 8 straps. However, lace-up ankle braces as well as braces employing such prior figure 8 straps commonly experience a similar problem, in that when a patient dorsiflexes his or her foot, such prior figure-8 straps gap. And when the patient plantarflexes his or her foot, such prior figure-8 straps overtighten, creating the danger for unstable ankle motion and/or a less consistently secure fit of the orthosis and/or its straps to the wearer through an entire potential range or motion of the ankle joint of the wearer. Accordingly, there remains a need for improved ankle orthoses to serve the needs of those suffering the growing number of ankle sprains and like injuries.

### SUMMARY

An ankle orthosis is provided. The orthosis is configured for attachment around an ankle of a wearer. The orthosis includes a fabric portion comprising closure flaps, the fabric portion configured to receive a lower leg of the wearer through the closable flaps. The orthosis includes a rigid upright assembly, wherein at least a portion of the rigid upright assembly is disposed within the fabric portion. The orthosis includes at least one adjustable cuff assembly configured to encircle the lower leg of the wearer above the ankle over the fabric portion and apply medial-lateral force to the lower leg of the wearer, thereby, preventing a tibia and fibula of the wearer from separating. The orthosis includes a first fastener coupled to a lateral side of the orthosis at a position inferior of the ankle joint of the wearer. The orthosis includes a first midfoot strap configured to wrap at least partly around the midfoot of the wearer over the fabric portion. The first midfoot strap includes a first end coupled to the same position on the lateral side of the orthosis as the first fastener, and a second end configured to couple to the first fastener.

In some embodiments the fabric portion includes a first portion having a desired amount of stretchability and including the closure flaps, which are disposed at an anterior of the fabric portion, wherein securing the closure flaps around the lower leg of the wearer forms a foot hole at the distal end of the fabric portion and a lower leg hole at the proximal end of the fabric portion. In some embodiments the fabric portion includes a heel hole disposed in a posterior portion of the fabric portion, between the foot hole and the lower leg hole. In some embodiments the fabric portion includes a second portion, having a lower degree of stretchability than the first portion, disposed on medial and lateral sides of the fabric portion and configured to encase at least a portion of the upright assembly. In some embodiments the fabric portion includes a posterior spine configured to be disposed along an Achilles tendon of the wearer and including a polypropylene webbing or seam tape.

In some embodiments, the rigid upright assembly includes an underfoot portion that bends to form lateral and medial lower uprights extending substantially perpendicular to the underfoot portion, a lateral upper upright having a proximal end pivotally coupled to a distal end of the lateral lower upright by a lateral joint, and a medial upper upright having a proximal end pivotally coupled to a distal end of the medial lower upright by a medial joint, the medal and lateral upper uprights each extending proximally to approximately half way between the ankle and the calf of the wearer when the orthosis is donned by the wearer.

In some embodiments the rigid upright assembly includes an underfoot portion that bends to form lateral and medial lower uprights extending substantially perpendicular to the underfoot portion, a medial upper upright having a distal end coupled to a proximal end of the medial lower upright by a medial joint, wherein a proximal end of the medial upper upright extends to the calf when the orthosis is donned by the wearer. The lateral lower upright extends, unjointed, to the calf when the orthosis is donned by the wearer, thereby providing a diagnosis-specific torsional affect opposing or resisting external rotation of the ankle of the wearer.

In some embodiments the rigid upright assembly includes an underfoot portion that bends to form lateral and medial lower uprights extending substantially perpendicular to the underfoot portion, a medial upper upright having a distal end coupled to a proximal end of the medial lower upright by a medial joint, wherein a proximal end of the medial upper upright extends to the calf when the orthosis is donned by the wearer, and a lateral upper upright having a distal end coupled to a proximal end of the lateral lower upright by a pivot structure including a plurality of fingers. Each finger includes a first portion coupled to and extending substantially straight away from the lower lateral upright and substantially, and a second portion extending from the first portion and having an oblong curvature that ultimately couples with the lateral upper upright. When the lateral upper upright is bent with respect to the lateral lower upright, the second portion of each of the fingers bends but remains in a common plane.

In some embodiments each of the at least one adjustable cuff assembly includes a posterior cuff portion configured to be disposed against posterior portions of the lower leg of the user above the ankle, a first anterior cuff portion pivotally coupled to a first end of the posterior cuff portion, a second anterior cuff portion slidingly coupled to a second end of the posterior cuff portion, and a third anterior cuff portion pivotally coupled to the second anterior cuff portion. The first, second and third cuff portions are configured to be disposed against respective portions of a side or anterior of the lower leg of the user above the ankle.

In some embodiments, the posterior cuff portion includes a post and the second anterior cuff portion includes a slot configured to slidingly receive the post. In some embodiments the slot extends substantially parallel to a length of circumferential extension of posterior cuff portion about the lower leg of the wearer. In some embodiments the slot is disposed toward an opposite end of the second anterior cuff portion from an end that is pivotally attached to the third anterior cuff portion.

In some embodiments at least one surface of the first anterior cuff portion includes a plurality of ratcheting teeth configured to be received by a complementary locking feature.

In some embodiments the third anterior cuff portion includes an aperture configured to receive at least a terminal end of the first anterior cuff therethrough.

In some embodiments the at least one adjustable cuff assembly includes a first adjustable cuff assembly configured to be disposed adjacent a proximal edge of the fabric portion and proximal of the ankle of the wearer, and a second adjustable cuff assembly configured to be disposed proximal of the ankle of the wearer and distal of the first adjustable cuff assembly.

In some embodiments the fabric portion is configured to encircle the lower leg of the wearer from a forefoot of the wearer to approximately half way between the ankle and a calf of the wearer. In some embodiments the fabric portion is configured to encircle the lower leg of the wearer from a forefoot of the wearer to a calf of the wearer.

In some embodiments, when properly secured around the lower leg of the wearer, the first midfoot strap extends from the first end, under the foot of the wearer, up a medial side of the midfoot of the wearer, over a top of the foot of the wearer distal of the ankle, through a slot or around a bar of the first fastener, and doubling back and securing to itself inferior of the ankle, thereby, providing adjustable support at the midfoot of the wearer.

In some embodiments the second end of the first midfoot strap includes at least one of hook and look fasteners and complementary snap fasteners.

In some embodiments the orthosis further includes a second fastener coupled to a medial side of the orthosis at a position inferior of the ankle joint of the wearer, and a second midfoot strap configured to wrap at least partly around the midfoot of the wearer over the fabric portion. The second midfoot strap includes a first end coupled to the same position on the medial side of the orthosis as the second fastener, and a second end configured to couple to the first fastener.

In some embodiments, when properly secured around the lower leg of the wearer, the second midfoot strap extends from the first end, under the foot of the wearer, up a lateral side of the midfoot of the wearer, over a top of the foot of the wearer distal of the ankle, through a slot or around a bar of the second fastener, and doubling back and securing to itself inferior of the ankle, thereby, providing adjustable support at the midfoot of the wearer.

In some embodiments, for at least one of the first and second midfoot straps, at least the portion of the strap configured to be disposed against an underside of the arch of the foot of the wearer has a first width and tapers to a second width that is narrower than the first width as the strap extends toward the second end.

In some embodiments the orthosis includes at least one of a lateral padded boarder coupled to and extending substantially along an entire lateral extent of the fabric portion, and a medial padded boarder coupled to and extending substantially along an entire medial extent of the fabric portion.

In some embodiments a method of utilizing an ankle orthosis configured for attachment to an ankle of a wearer is provided. The method includes receiving at least a portion of a lower leg anteriorly into a fabric portion of the ankle orthosis. The method includes tightening at least one strap around the fabric portion with the lower leg of the wearer secured therein. The method includes tightening at least one adjustable cuff assembly around the fabric portion with the lower leg of the wearer secured therein, thereby preventing a tibia and fibula of the wearer from separating. In some embodiments of such a method, the orthosis is an orthosis as described anywhere in this disclosure.

In some embodiments, a method of manufacturing an ankle orthosis configured for attachment around an ankle of a wearer is provided. The method includes providing a fabric portion including closure flaps, the fabric portion configured to receive a lower leg of the wearer through the closable flaps. The method includes sewing or otherwise encapsulating at least a portion of a rigid upright assembly into the fabric portion. The method includes providing and/or assembling at least a first adjustable cuff assembly configured to encircle the lower leg of the wearer above the ankle over the fabric portion and apply medial-lateral force to the lower leg of the wearer to, thereby, prevent a tibia and fibula of the wearer from separating. The method includes coupling a first fastener to a lateral side of the orthosis at a position inferior of the ankle joint of the wearer. The method includes coupling a first end of a first midfoot strap to the same position on the lateral side of the orthosis as the first fastener.

In some embodiments, the method further includes coupling a second fastener to a lateral side of the orthosis at a position inferior of the ankle joint of the wearer, and coupling a first end of a second midfoot strap to the same position on the medial side of the orthosis as the second fastener. In some such methods, the orthosis is an orthosis as described anywhere in this disclosure.

### BRIEF DESCRIPTION OF THE DRAWINGS

FIG. 1 illustrates a front elevated view of a brace disposed on a wearer's lower leg, according to some example embodiments;

FIG. 2 illustrates a side view of the brace of FIG. 1, according to some example embodiments;

FIG. 3 illustrates a posterior view of the brace of FIG. 1, according to some example embodiments;

FIG. 4 illustrates a perspective view of the brace of FIG. 1, according to some example embodiments;

FIG. 5 illustrates the fabric component of the brace of FIG. 1, according to some example embodiments;

FIG. 6 illustrates an upright assembly of the brace of FIG. 1, according to some example embodiments;

FIG. 7 illustrates an optional foot plate for any brace described herein, according to some example embodiments;

FIG. 8 illustrates a side view of a single-anchor adjustable cuff assembly, according to some example embodiments;

FIG. 9 illustrates a top view of the single-anchor adjustable cuff assembly of FIG. 8, according to some example embodiments;

FIG. 10 illustrates another top view of anterior and posterior cuff portions of the cuff assembly of FIG. 8, according to some example embodiments;

FIG. 11 illustrates perspective views of slotted cuffs of respective adjustable slotted cuff assemblies, according to some example embodiments;

FIG. 12 illustrates perspective views of the slotted cuffs of FIG. 11 engaged to one another;

FIG. 13 illustrates a front elevated view of any short brace described herein utilizing an adjustable slotted cuff assembly having a medial reel-and-lace system, according to some example embodiments;

FIG. 14 illustrates a medial side view of the brace of FIG. 13;

FIG. 15 illustrates a lateral side view of the brace of FIG. 13;

FIG. 16 illustrates a medial side view of the brace of FIG. 14 inserted in a shoe of the wearer;

FIG. 17 illustrates a front elevated view of any short brace described herein utilizing an adjustable slotted cuff assembly having a posterior reel-and-lace system, according to some example embodiments;

FIG. 18 illustrates a lateral side view of the brace of FIG. 17;

FIG. 19 illustrates a posterior view of the brace of FIG. 17 disposed in a shoe of the wearer;

FIG. 20 illustrates a medial side view of the brace of FIG. 17 disposed in a shoe of the wearer;

FIG. 21 illustrates a front elevated view of another embodiment of a brace disposed on a wearer's lower leg, according to some example embodiments;

FIG. 22 illustrates a side view of the brace of FIG. 21, according to some example embodiments;

FIG. 23 illustrates a bottom view of the brace of FIG. 21, according to some example embodiments;

FIG. 24 illustrates a side view of the brace of FIG. 21 with a shoe on the wearer's foot and over the brace, according to some example embodiments;

FIG. 25 illustrates a front view of another embodiment of a brace, according to some example embodiments;

FIG. 26 illustrates a front elevated view of the brace of FIG. 25 disposed on a wearer's foot, according to some example embodiments;

FIG. 27 illustrates a bottom view of the brace of FIG. 25, according to some example embodiments;

FIG. 28 illustrates a side view of the brace of FIG. 25 on the wearer's foot, according to some example embodiments;

FIG. 29 illustrates a side view of the brace of FIG. 25 on the wearer's foot and over the brace, according to some example embodiments;

FIG. 30 illustrates a front elevated view of an embodiment of a tall brace, according to some example embodiments;

FIG. 31 illustrates a side view of the brace of FIG. 30;

FIG. 32 illustrates a bottom view of the brace of FIG. 30 disposed on a wearer's lower leg, according to some example embodiments;

FIG. 33 illustrates an elevated front view of the brace of FIG. 30 disposed on a wearer's lower leg, according to some example embodiments;

FIG. 34 illustrates a side view of the brace of FIG. 33;

FIG. 35 illustrates a side view of the brace of FIG. 30 further disposed in a shoe of the wearer;

FIG. 36 illustrates an elevated front view of the brace of FIG. 36;

FIG. 37 illustrates an upright assembly for any tall brace disclosed herein, according to some example embodiments;

FIG. 38 illustrates another upright assembly for any tall brace disclosed herein, according to some example embodiments;

FIG. 39 illustrates the upright assembly of FIG. 38 substantially flipped along the horizontal compared to FIG. 38;

FIG. 40 illustrates a perspective view of the upright assembly of FIG. 39 in a substantially unbent orientation, according to some example embodiments;

FIG. 41 illustrates a perspective view of the upright assembly of FIG. 39 in a substantially bent orientation;

FIG. 42 illustrates a substantially frontal view of the upright assembly of FIG. 41 in the substantially bent orientation;

FIG. 43 illustrates a front elevated view of yet another embodiment of a tall brace, according to some example embodiments;

FIG. 44 illustrates a posterior view of the brace of FIG. 43;

FIG. 45 illustrates a side view of the brace of FIG. 43 disposed on a wearer's lower leg, according to some example embodiments;

FIG. 46 illustrates an elevated front view of the brace of FIG. 45;

FIG. 47 illustrates an elevated front view of the brace of FIG. 40 disposed in a shoe of the wearer;

FIG. 48 illustrates a side view of the brace of FIG. 47;

FIG. 49 illustrates a front elevated view of yet another embodiment of a tall brace, according to some example embodiments;

FIG. 50 illustrates a side view of the brace of FIG. 49;

FIG. 51 illustrates a front elevated view of the brace of FIG. 49 with a wearer's lower leg disposed therein;

FIG. 52 illustrates a side view of the brace of FIG. 51;

FIG. 53 illustrates a front elevated view of the brace of FIG. 51 disposed in a shoe of the wearer, according to some example embodiments;

FIG. 54 illustrates a side view of the brace of FIG. 53;

FIG. 55 illustrates a front view of yet another embodiment of a brace, according to some example embodiments;

FIG. 56 illustrates a side view of braces having midfoot strap fasteners and corresponding midfoot straps coupled to the brace at different locations on the brace, according to some example embodiments;

FIG. 57 illustrates a side view of braces having midfoot strap fasteners and corresponding midfoot straps both coupled to the brace at a same location on the brace, according to some example embodiments;

FIG. 58 illustrates a portion of one of the braces of FIG. 57 but viewed from the opposite side;

FIG. 59 illustrates yet another adjustable cuff assembly, according to some example embodiments;

FIG. 60 illustrates the adjustable cuff assembly of FIG. 59 but viewed from the opposite side;

FIG. 61 illustrates the adjustable cuff assembly of FIGS. 59-60 disposed a around a portion the brace of FIG. 55;

FIG. 62 illustrates a front view of a tall brace having midfoot strap fasteners and corresponding midfoot straps both coupled to the brace at a same location on the brace, according to some example embodiments;

FIG. 63 illustrates a rear view of a portion of the brace of FIG. 62;

FIG. 64 illustrates a top view of the brace of FIG. 62;

FIG. 65 illustrates a side view of a portion of the brace of FIG. 62;

FIG. 66 illustrates another side view of a portion of the brace of FIG. 62;

FIG. 67 illustrates a flowchart of a method of utilizing an ankle brace in the treatment of ankle injuries, in accordance with some embodiments; and

FIG. 68 illustrates a flowchart of a method of manufacture of an ankle brace, in accordance with some embodiments.

### DETAILED DESCRIPTION

Embodiments of this disclosure relate to ankle braces for use in treating a variety of injuries to the ankle joint and/or surrounding tissues, for example and not limitation, ankle sprains. More specifically, the present disclosure contemplates multiple embodiments of a short brace 100, and multiple embodiments of a relatively taller brace 200, each comprising features as described in connection with any figure(s) below.

### Short Brace Embodiments

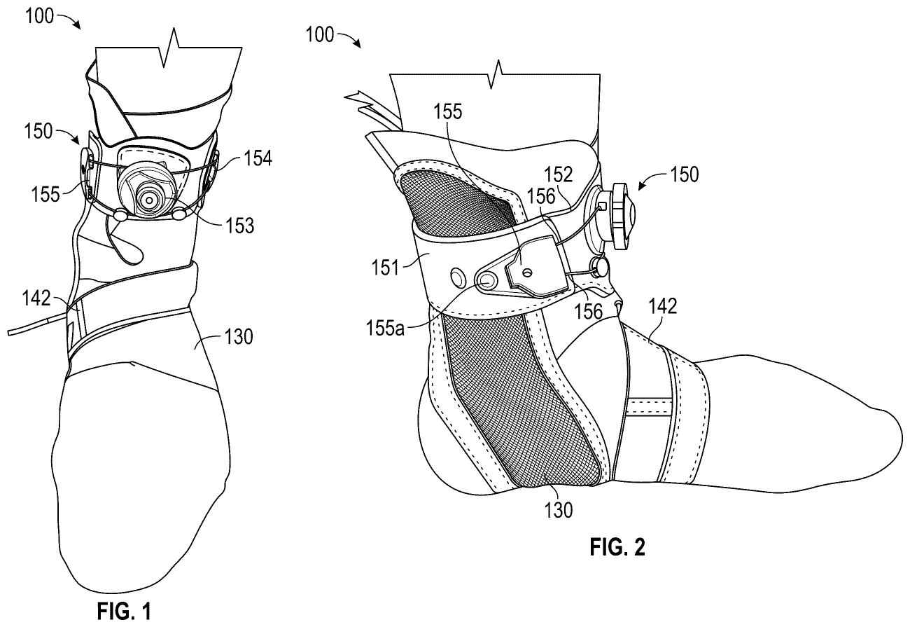

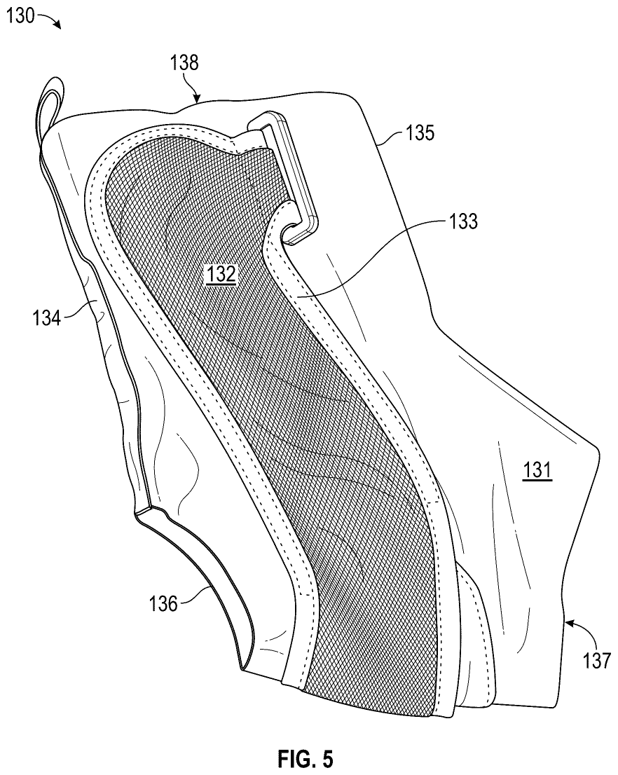

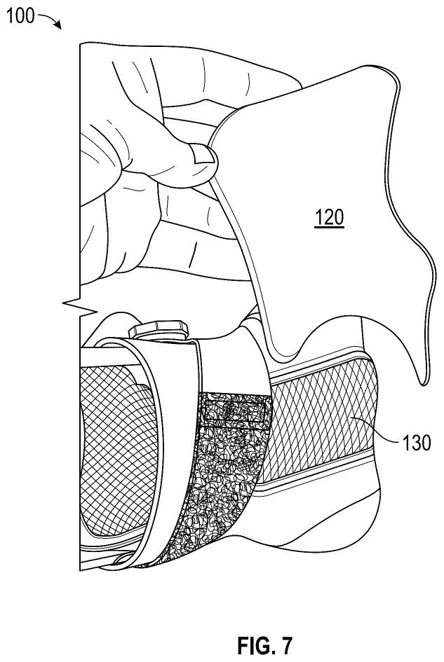

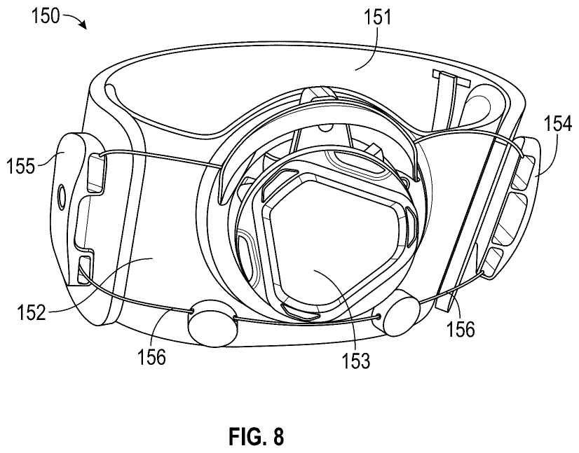

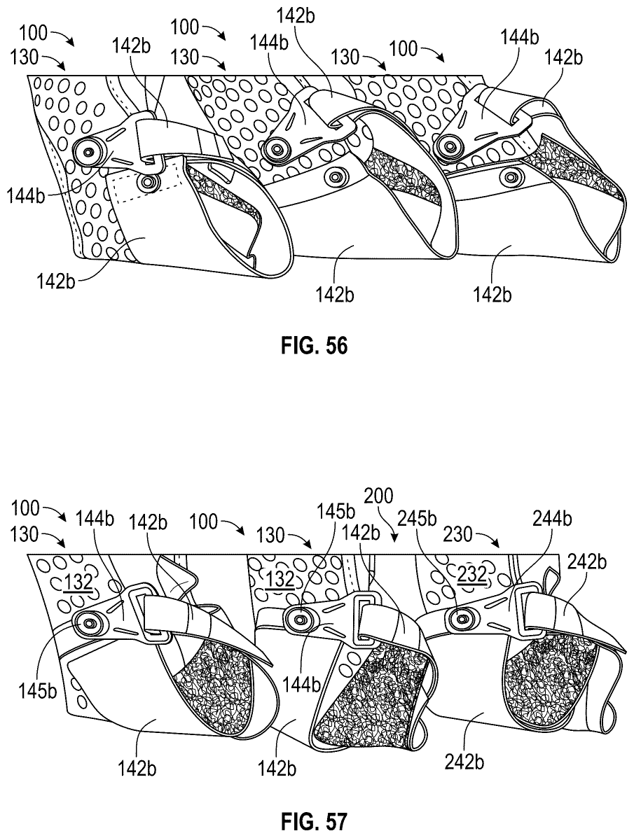

FIGS. 1-29 and 55-61 illustrate various views of several embodiments of a short ankle brace 100. While not limited to such uses, such short braces 100 may be utilized to treat chronic ankle instability, medial and/or lateral instabilities, and for prophylactic use. Such short braces 100 may be configured to prevent or substantially reduce foot inversion, foot eversion, tibia-fibula separation, foot pronation and/or foot supination. In some embodiments, a brace 100 according to at least one of FIGS. 1-29 and 55-61 includes one or more of: a midfoot strap 142 and corresponding fastener 144, a unique “figure 8” strapping assembly comprising first and second straps 142a, 142b, similar to midfoot strap 142 but mounted in opposite medial-lateral orientations to one another on brace 100, and configured to be secured, below, inferior to or distal of an ankle joint of the wearer, to respective fasteners 144a, 144b; a compression cuff 150, 160, 170, 180, or 190 configured to prevent a tibia and/or fibula of the wearer from separating relative to one another while brace 100 is worn; an articulated upright assembly 110 embedded in a fabric portion 130 (see, e.g., soft goods shown in FIG. 5), wherein adjustment of the compression cuff 150, 160, 170, 180, or 190 and straps 142, 143, and/or 142a, 142b simultaneously provide a rigid frame, a frame cover, and a soft goods interface with the lower leg of the wear.

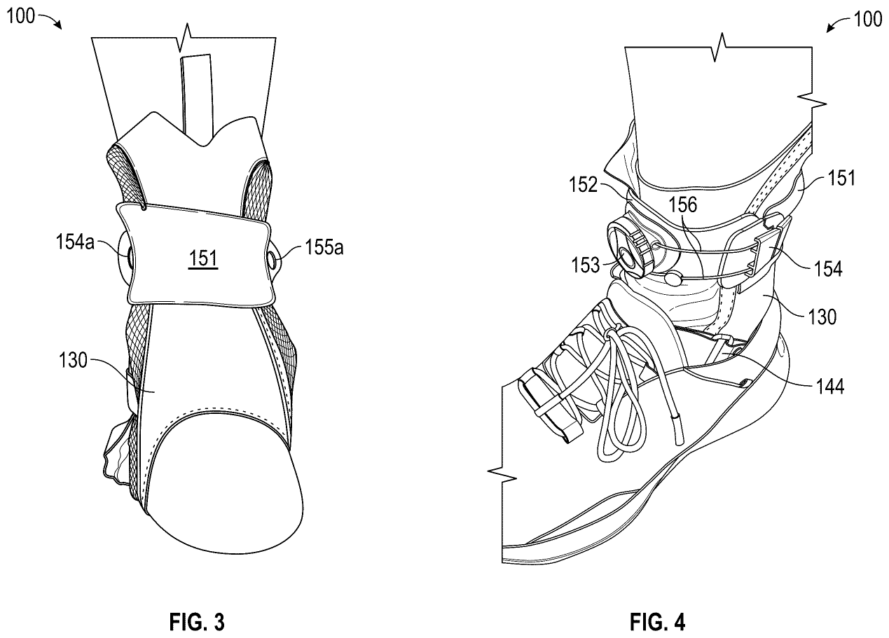

FIGS. 1-4 illustrate several views of an embodiment of a short brace 100. Specifically, FIG. 1 illustrates a front elevated view of a brace 100 disposed on a wearer's lower leg, according to some example embodiments. FIG. 2 illustrates a side view of brace 100 of FIG. 1. FIG. 3 illustrates a posterior view of brace 100 of FIG. 1. And FIG. 4 illustrates a perspective view of brace 100 of FIG. 1.

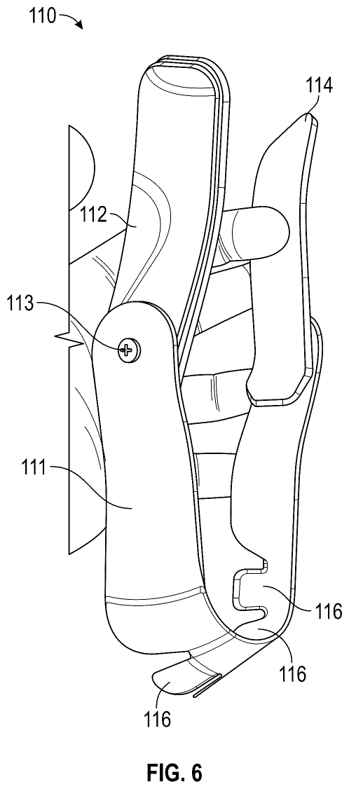

Brace 100 comprises a fabric portion 130 (e.g., a closeable tubular, sleeve as shown in FIG. 5) having a rigid upright assembly 110 (see, e.g., FIG. 6) disposed, or sewn, at least partially therein. For example, FIG. 5 illustrates fabric portion 130 configured to receive the lower leg of the wearer into fabric portion 130 from an anterior thereof. As shown, fabric portion 130 may comprise a stretchy portion 131 (e.g., a first portion having a desired amount of stretchability, e.g., a material and/or fabric configured to stretch, such as nylon) comprising hook and loop front closure flaps 135 configured to receive the lower leg of the wearer therein through the anterior of fabric portion 130 when flaps 135 are open. In some embodiments, stretchy portion 131 comprises substantially an entirety of the base layer of fabric portion 130. Flaps 135 are then configured to couple to one another, thereby enclosing the lower leg of the wearer in fabric portion 130. When so secured, fabric portion 130 comprises a foot hole at the distal end thereof, a lower leg hole at the proximal end thereof, and a heel hole 136 in a posterior portion thereof, between the foot hole and the lower leg hole. Fabric portion 130 further comprises a less stretchy portion 132 (e.g., a second portion having a lower degree of stretchability compared to the first, stretchy portion 131, e.g., in some embodiments, a material and/or fabric configured to substantively not stretch such as teryene mesh), disposed on medial and lateral sides thereof. Less stretchy portion 132 is configured to extend along the underside and both the lateral and medial sides of fabric portion 130 and encircle or encase at least a portion of upright assembly 110 therein and/or between portions 131 and 132 (see, e.g., FIGS. 5 and 6). In some embodiments, less stretchy portion 132 comprises an edge finisher (e.g., teryene grograin binding 133) that is, then, sewn to stretchy portion 131. In some embodiments, a posterior spine (e.g., from heel hole 136 to the posterior upper (proximal) edge) of fabric portion 130 may comprise a polypropylene webbing 134 for added comfort and/or stretch compliance along the Achilles tendon of the wearer. In some embodiments, webbing 134 may comprise a seam tape. In some embodiments, webbing 134 may comprise a material with a relatively increased ability to stretch compared to polypropylene, such as polyspandex, or in some such embodiments, a polyspandex seam tape. In some embodiments, fabric portion 130 comprises a finger loop (e.g., a polypropylene finger loop) configured to aid the wearer's donning of brace 100.

FIG. 6 illustrates upright assembly 110 of brace 100 as mentioned above. At least a portion of upright assembly 110 is sewn into fabric portion 130. Upright assembly 110 comprises an underfoot portion 116, which bends and then extends from both ends, substantially perpendicularly of underfoot portion 116, to form lateral and medial lower uprights 111. In some embodiments, underfoot portion 116 and lateral and medial uprights 111 are monolithic, formed as a single piece that may be bent or otherwise formed into a substantially “U” shaped form, thereby decreasing part count of brace 100. In some embodiments, underfoot portion 116 is formed having a plurality of tabs with material portions of the single piece between missing, removed or molded to so as to form individual tabs that allow the monolithic portion comprising underfoot portion 116 and lateral and medial uprights 111 to bend into the above-described “U”-shaped form. A distal end of a first upper upright 112 is rotatably or pivotally coupled to a proximal end of one of the lateral and medial lower uprights 111 via joint 113, which may comprise an axle, rivet or similar feature. Similarly, a distal end of a second upper upright 114 is rotatably or pivotally coupled to a proximal end of the other of the lateral and medial lower uprights 111 via a joint (not shown in FIG. 6) similar to joint 113. Accordingly, as shown, both the medial and lateral uprights of upright assembly 110 are articulated. Upright assembly 110 and/or its components may be sufficiently rigid to provide support to the lower leg of the wearer, comprising, for example and not limitation, aluminum, stainless steel, carbon fiber, a rigid plastic, or another composite construction.

Turning back to FIGS. 1-4, brace 100 comprises midfoot strap 142 and corresponding fastener 144. Midfoot strap 142 is configured to wrap at least partially around a midfoot (e.g., the arch area) of the wearer over an outside of fabric portion 130 of brace 100. Both fastener 144 and a first end of midfoot strap 142 may be coupled to a lateral side or bottom of fabric portion 130 at positions that are both inferior to or distal of the ankle of the wearer, or to a similar portion of upright assembly 110 disposed therein (e.g., to medial and/or lateral upright 111). Strap 142 may extend from this first fixed end, under the foot of the wearer, up a medial side of the midfoot of the wearer, over a top of the foot of the wearer inferior to or distal of the ankle joint, through a slot or around a bar of fastener 144, double back and secure to itself, thereby, providing adjustable support at the midfoot of the wearer. Accordingly, a second end of strap 142 may comprise hook and loop fasteners or complementary snap (e.g., snap-button) fasteners to facilitate the above-described self-securing. Fastener 144 may comprise a rivet and ring arrangement, such as a d-ring, plastic chafe and/or fabric chafe, coupled to the lateral side of fabric portion 130, or similar position on upright assembly 110 disposed therein, inferior to or distal of the ankle of the wearer.

In some embodiments, brace 100 further comprise an adjustable cuff assembly 150, 160, 170, 180, or 190 which is configured to encircle the lower leg of the wearer over fabric portion 130 and apply medial-lateral force to the lower leg of the wearer to, thereby, prevent a tibia and/or fibula of the wearer from separating relative to one another while brace 100 is worn. Several different examples of such adjustable cuff assemblies are described in connection with FIGS. 8-20 and/or 55 and 59-66 below.

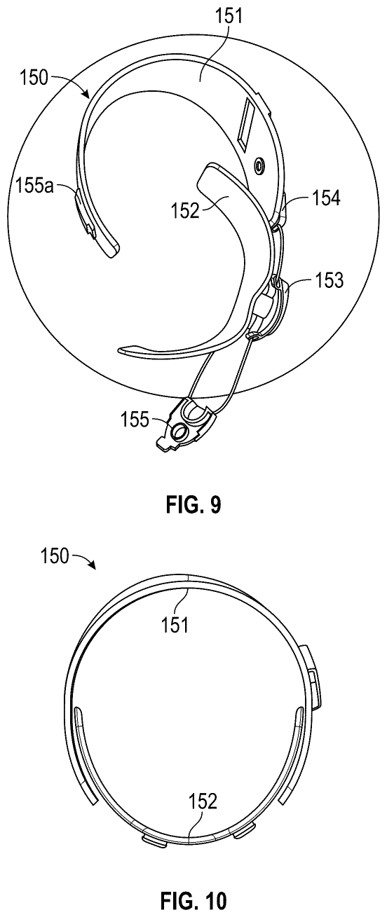

For example, FIGS. 8-10 show a first embodiment of a single-anchor adjustable cuff assembly 150 configured to encircle the lower leg of the wearer above the ankle and over fabric portion 130 and apply medial-lateral force to the lower leg of the wearer to, thereby, prevent a tibia and/or fibula of the wearer from separating relative to one another while brace 100 is worn. Specifically, FIG. 8 illustrates a side view of single-anchor adjustable cuff assembly 150. FIG. 9 illustrates a top view of assembly 150 of FIG. 8. And FIG. 10 illustrates another top view of anterior and posterior cuff portions 151, 152 of assembly 150 of FIG. 8.

Cuff assembly 150 comprises a posterior cuff portion 151, an anterior cuff portion 152, and a reel-and-lace system coupled between posterior cuff portion 151 and anterior cuff portion 152. In some embodiments, terminal ends of anterior cuff portion 152 may be configured to fit (e.g., nest) within the perimeter of the terminal ends of posterior cuff portion 151 such that, when the reel-and-lace system is tightened, anterior cuff portion 152 is disposed against anterior and side portions of the lower leg of the user above the ankle joint and posterior cuff portion 151 is disposed against posterior and side portions of the lower leg of the user above the ankle joint, and over at least the ends of anterior cuff portion 152. In some embodiments, padding is provided on an inside surface of one or both of posterior cuff portion 151 and anterior cuff portion 152.

The reel-and-lace system comprises a reel 153 coupled to an anterior surface of anterior cuff portion 152, a first anchor point 154 disposed on one terminal end of posterior cuff portion 151, a second anchor point 155 disposed on the other terminal end of posterior cuff portion 151, and a lace 156 configured to wrap around reel 153 and extend through apertures or guides in each of first and second anchor points 154, 155. While the present disclosure is not so limited, in some embodiments, such reel-and-lace systems may comprise a Boa reel, which for example, may provide various models providing various powers or ability to compress cuff portions 151,152. Examples include, but are not limited to, Boa's L6 (e.g., low power gen 6) Boa's Medium power 160, which by comparison is more powerful but bulkier, or Boa's L+1 reel which provides power between that of the L6 and Boa's medium power option and a balance between power and size/profile. At least one of first and second anchor points 154, 155 is detachable from a base 155a disposed on the corresponding terminal end of posterior cuff portion 151 (e.g., anchor point 155 as shown detached from base 155a in FIG. 9). Accordingly, anterior cuff portion 152 hinges, or is free swinging, from posterior cuff portion 151. Cuff assembly 150 may be applied over fabric portion 130 of worn brace 100 from the posterior of fabric portion 130 and of brace 100. In other words, worn brace 100 (or at least fabric portion 130 and upright assembly 110 disposed at least partially therein) may be disposed within the open cuff assembly 150 from the anterior of assembly 150, anchor point 155 may be resecured to base 155a, and reel 153 may be rotated until lace 156 imparts sufficient tension between reel 153 and anchor points 154, 155 to pull anterior cuff portion 152 and posterior cuff portion 151 together and apply the desired amount of medial-lateral force to the lower leg of the wearer to prevent the tibia and/or fibula of the wearer from separating relative to one another while brace 100 is worn.

It should be noted that the current disclosure also contemplates use of double-anchor embodiments of assembly 150 where both first and second anchor points 154, 155 are detachable from respective bases 154a, 155a, disposed on respective terminal ends of posterior cuff portion 151. Accordingly, when both anchor points 154 and 155 are detached from their detachable bases 154a, 155a, anterior cuff portion 152 would, contrarily, be completely separable from posterior cuff portion 151.

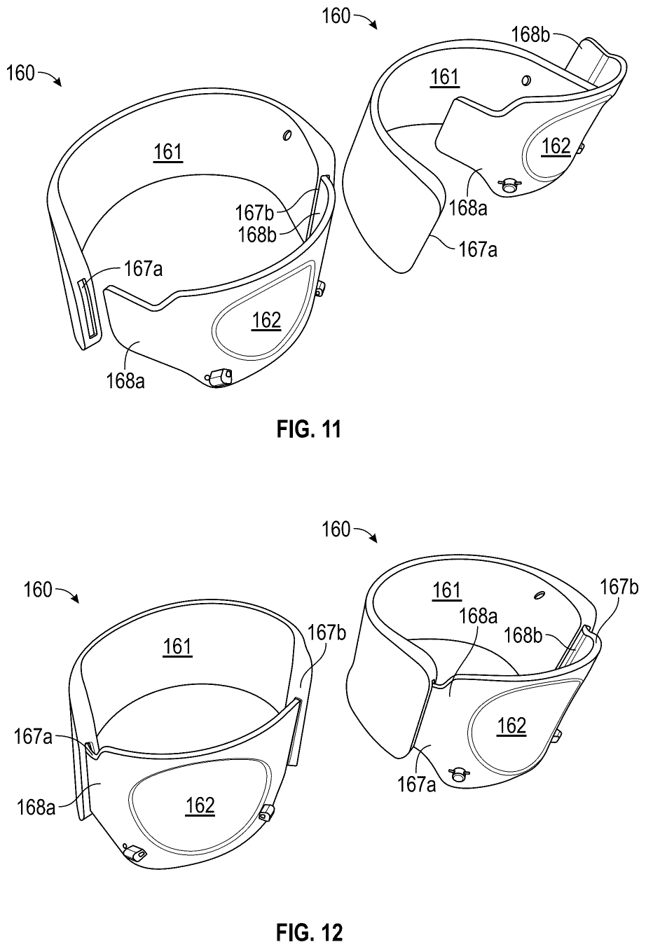

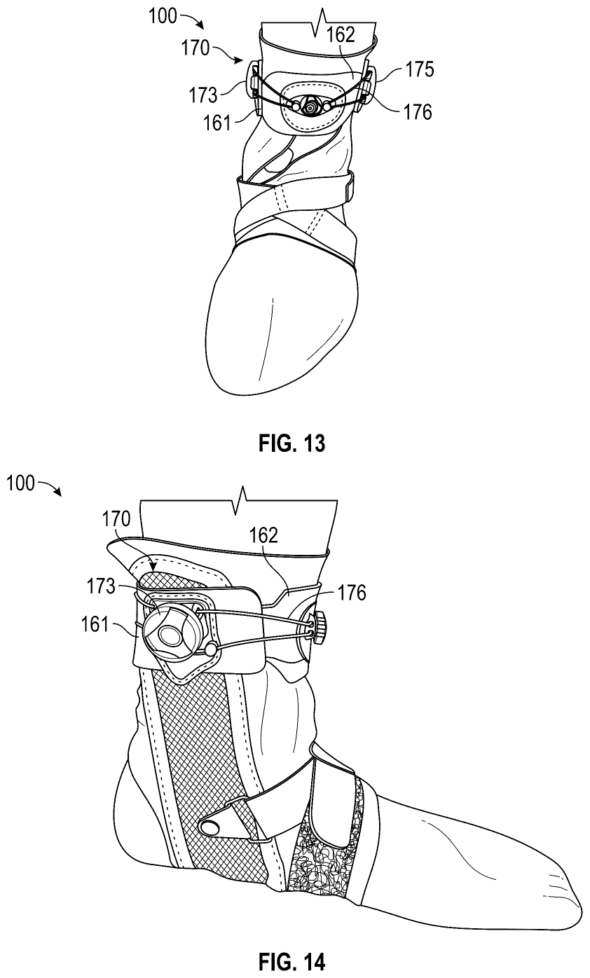

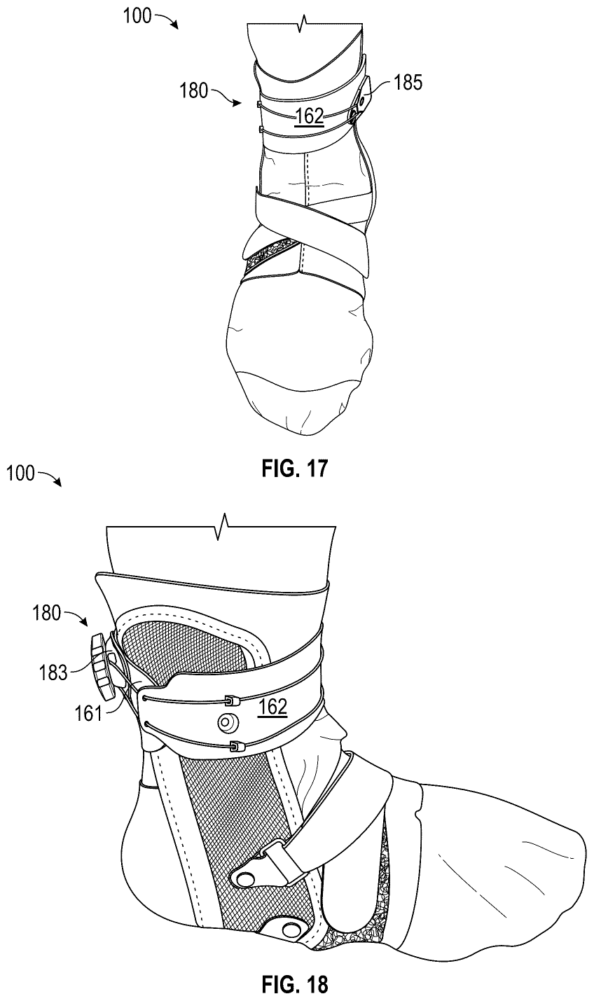

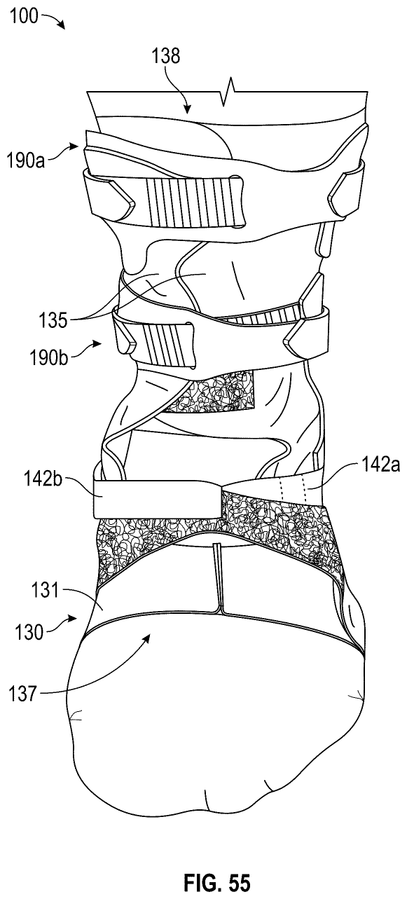

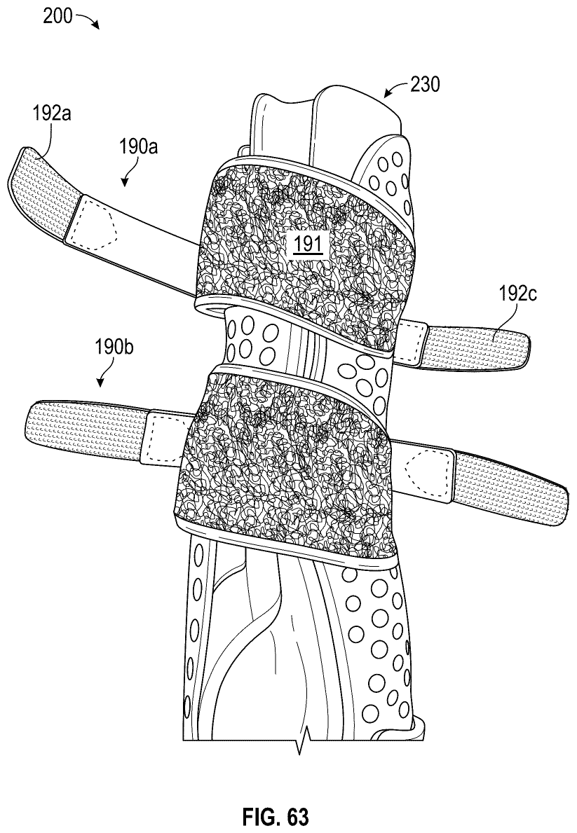

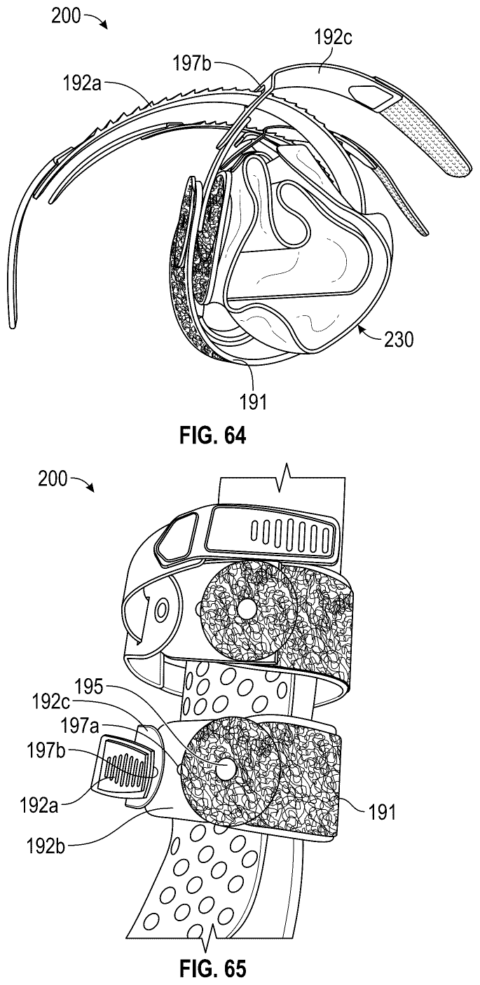

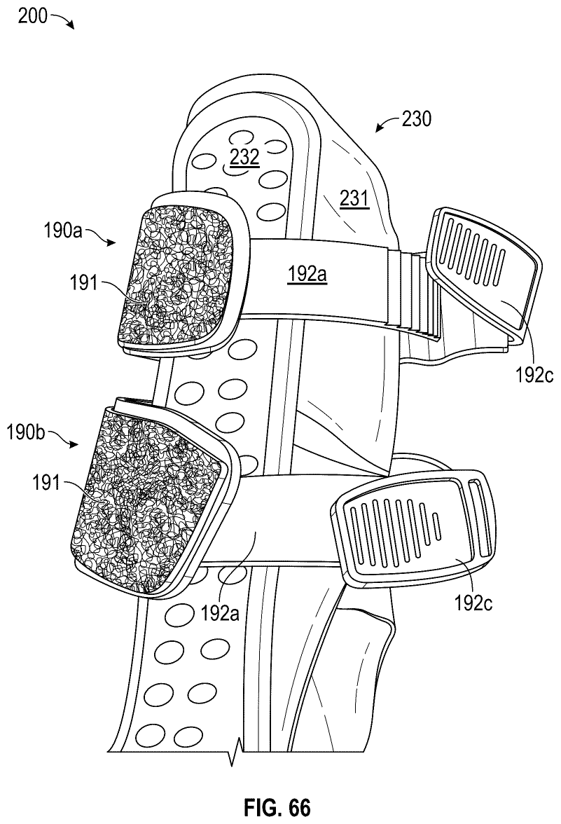

Yet other adjustable cuff assembly embodiments are also contemplated for use with any brace 100, 200 described in this disclosure. For example, FIGS. 11 and 12 illustrate alternative slotted cuff portions 161 and 162 for use in an adjustable cuff assembly 160 having an anterior reel-and-lace system (similar to that previously shown in FIGS. 8-10), in an adjustable cuff assembly 170 having a medial reel-and-lace system (as shown in FIGS. 13-16), or for use in an adjustable cuff assembly 180 having a posterior reel-and-lace system (as shown in FIGS. 17-20). Further, FIGS. 55 and 59-66 illustrate yet another adjustable cuff assembly 190 having slidable, rotatable and/or ratchetable cuff portions, as will be described in more detail later in this disclosure.

Where slotted cuff portions 161 and 162 are utilized in an anterior reel-and-lace system (as previously shown in FIGS. 8-10), such adjustable cuff assemblies may comprise substantially the same components as previously described in connection with FIGS. 8-10 except utilizing slotted cuff portions 161 and 162 rather than nesting cuff portions 151, 152.

As shown in FIGS. 11 and 12, posterior cuff portion 161 comprises respective slots 167a, 167b disposed in terminal ends of posterior cuff portion 161. Each terminal end 168a, 168b of anterior cuff portion 162 is configured to fit into a corresponding one of slots 167a, 167b of posterior cuff portion 161. Accordingly, terminal ends 168a, 168b of anterior cuff portion 162 are guided within slots 167a, 167b of posterior cuff portion 161 as the assembly is tightened, rather than being disposed against fabric layer 130 and potentially digging into the lower leg of the wearer. FIG. 11 illustrates anterior cuff portion 162 separated from posterior cuff portion 161, while FIG. 12 illustrates terminal ends 168a, 168b of anterior cuff portion 162 inserted into corresponding slots 167a, 167b of posterior cuff portion 161.

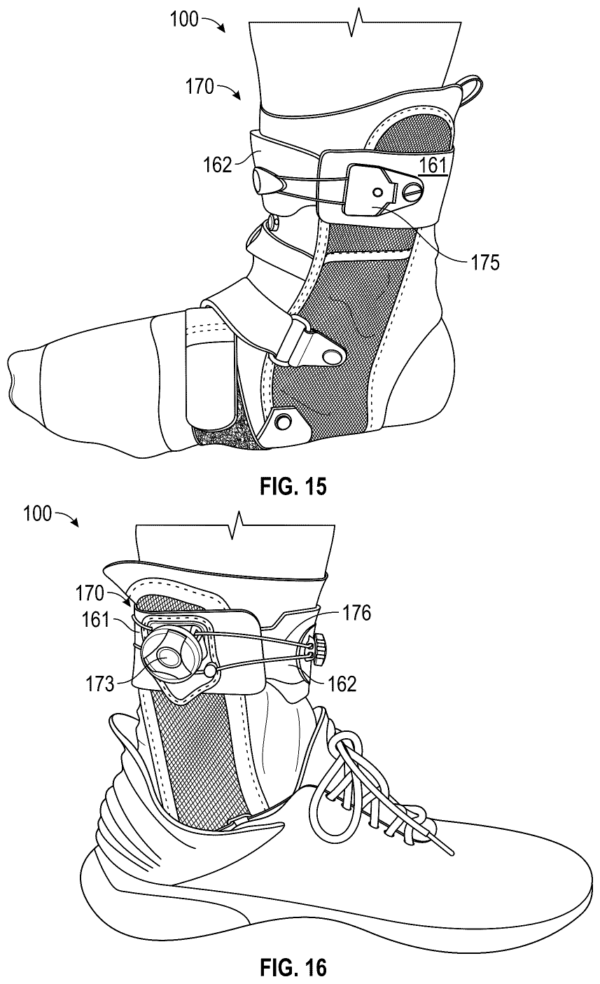

Turning to adjustable slotted cuff assemblies having a medial reel-and-lace system, FIG. 13 illustrates a front elevated view of any short brace 100 described herein alternatively utilizing an adjustable slotted cuff assembly 170 having a medial reel-and-lace system. FIG. 14 illustrates a medial side view of brace 100 of FIG. 13. FIG. 15 illustrates a lateral side view of brace 100 of FIG. 13. And FIG. 16 illustrates a medial side view of brace 100 of FIG. 14 inserted in a shoe of the wearer. The medial reel-and-lace system comprises a reel 173 coupled to a medial surface of posterior cuff portion 161, an anchor point 175 disposed on the other terminal end of posterior cuff portion 161, and a lace 176 configured to wrap around reel 173 and extend through apertures or guides on anterior cuff portion 162 and anchor point 175. Anchor point 175 is detachable from a base 175a (not visible in FIG. 15, but located under anchor point 175) disposed on the corresponding terminal end of posterior cuff portion 161. Accordingly, anterior cuff portion 162 hinges, or is free swinging, from posterior cuff portion 161. Cuff assembly 170 may be applied over fabric portion 130 of worn brace 100 from the posterior of fabric portion 130 and of brace 100. In other words, worn brace 100 (or at least fabric portion 130 and upright assembly 110 disposed at least partially therein) may be disposed within the open cuff assembly 170 from the anterior of assembly 170, anchor point 175 may be resecured to base 175a, and reel 173 may be rotated until lace 176 imparts sufficient tension between reel 173, anterior cuff portion 162 and anchor point 175 to pull anterior cuff portion 162 and posterior cuff portion 161 together and apply the desired amount of medial-lateral force to the lower leg of the wearer to prevent the tibia and/or fibula of the wearer from separating relative to one another while brace 100 is worn.

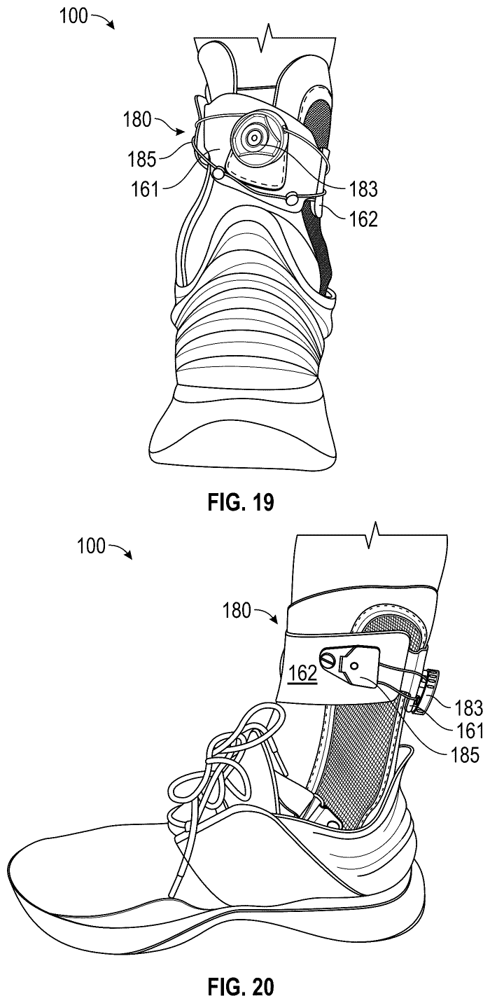

Turning to adjustable slotted cuff assemblies having a posterior reel-and-lace system, FIG. 17 illustrates a front elevated view of any short brace 100 described herein utilizing an adjustable slotted cuff assembly 180 having a posterior reel-and-lace system. FIG. 18 illustrates a lateral side view of brace 100 of FIG. 17. FIG. 19 illustrates a posterior view of brace 100 of FIG. 17 disposed in a shoe of the wearer. And FIG. 20 illustrates a medial side view of brace 100 of FIG. 17 disposed in a shoe of the wearer. The posterior reel-and-lace system comprises a reel 183 coupled to a posterior surface of posterior cuff portion 161, an anchor point 185 disposed on one terminal end of posterior cuff portion 161, and a lace 186 configured to wrap around reel 183 and extend through apertures or guides on anterior cuff portion 162 and anchor point 185. Anchor point 185 is detachable from a base 185a (not visible in FIG. 17 or 20, but located under anchor point 185) disposed on the corresponding terminal end of posterior cuff portion 161. Accordingly, anterior cuff portion 162 hinges, or is free swinging, from posterior cuff portion 161. Cuff assembly 180 may be applied over fabric portion 130 of worn brace 100 from the posterior of fabric portion 130 and of brace 100. In other words, worn brace 100 (or at least fabric portion 130 and upright assembly 110 disposed at least partially therein) may be disposed within the open cuff assembly 180 from the anterior of assembly 180, anchor point 185 may be resecured to base 185a, and reel 183 may be rotated until lace 186 imparts sufficient tension between reel 183, anterior cuff portion 162 and anchor point 185 to pull anterior cuff portion 162 and posterior cuff portion 161 together and apply the desired amount of medial-lateral force to the lower leg of the wearer to prevent the tibia and/or fibula of the wearer from separating relative to one another while brace 100 is worn. While brace 100 shown in FIGS. 17-21 may be configured for posterior entry of the wearer's lower leg into brace 100, the present disclosure is not so limited. It is also contemplated that brace 100, as shown in FIGS. 17-21 may alternatively be configured for anterior entry of the wearer's lower leg into brace 100.

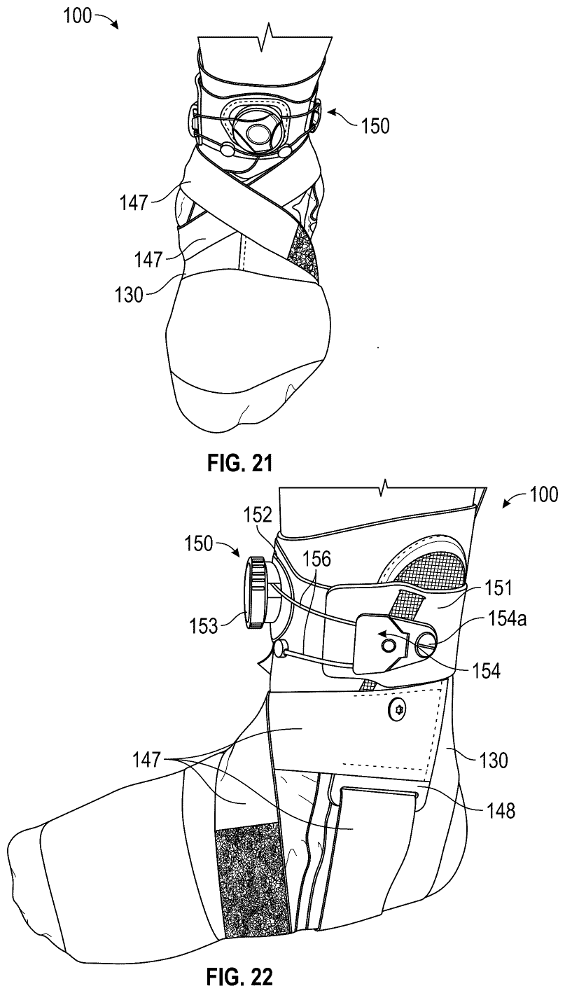

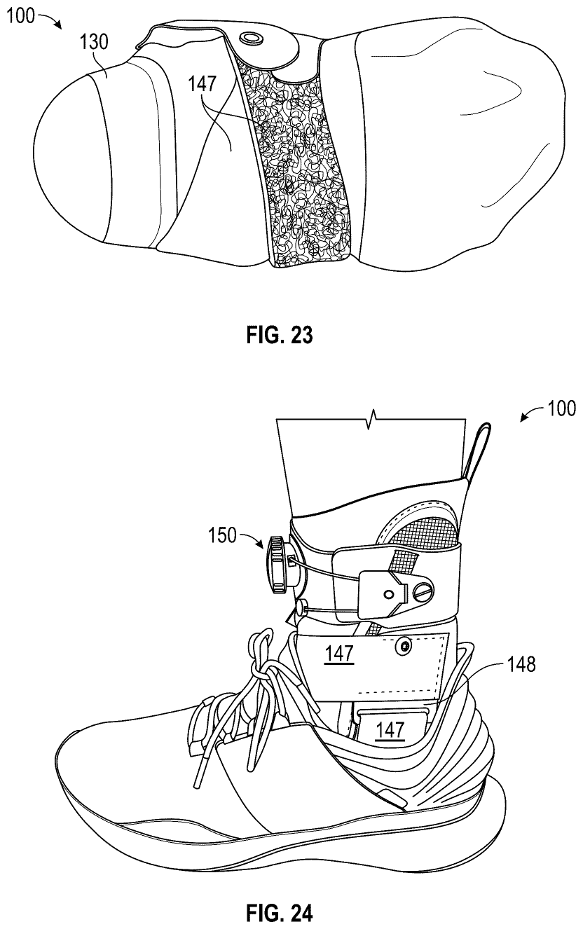

FIGS. 21-24 illustrate another embodiment of a short brace 100. In comparison to brace 100 of FIGS. 1-4, brace 100 of FIG. 21-24 omits midfoot strap 142 and corresponding fastener 144, instead, adding a single figure-8 strap 147 and corresponding fastener 148. Specifically, FIG. 21 illustrates a front elevated view of such a brace 100 disposed on a wearer's lower leg. FIG. 22 illustrates a side view of brace 100 of FIG. 21. FIG. 23 illustrates a bottom view of brace 100 of FIG. 21. And FIG. 24 illustrates a side view of brace 100 of FIG. 21 with a shoe on the wearer's foot and over brace 100.

A first end of single figure-8 strap 147 is coupled to a lateral side of fabric portion 130 and/or to lower lateral upright 111, lateral upper upright 112 or joint 113. Strap 147 extends from this lateral coupling point, over an anterior of the ankle joint below double-anchor adjustable cuff assembly 160, down a medial side of the foot of the wearer (e.g., at approximately the midfoot) around the underside of the midfoot of the wearer, up a lateral side of the midfoot of the wearer, over a top of the midfoot of the wearer, back down the medial side of the foot of the wearer, at least partially over the first such medial side pass, around the underside of the midfoot of the wearer, up the lateral side of the foot of the wearer, slightly posterior of the first such lateral side pass, extending through a slot, or around a bar, of fastener 148, doubling back and securing to itself, thereby, providing adjustable support along substantially the entire ankle of the wearer. Accordingly, the free end of strap 147 may comprise hook and loop fasteners or complementary snap (e.g., snap-button) fasteners for such self-securing. Fastener 148 may comprise a rivet and ring arrangement, such as a d-ring plastic chafe and/or fabric chafe, coupled to the lateral side of brace 100, for example, to fabric portion 130 and/or, lower lateral upright 111, lateral upper upright 112 or joint 113, of upright assembly 110 disposed therein (see, e.g., FIG. 22). However, such embodiments may still suffer from the varying gapping and/or over-tensioning in strap 147 when the wearer's foot is dorsiflexed and/or plantarflexed.

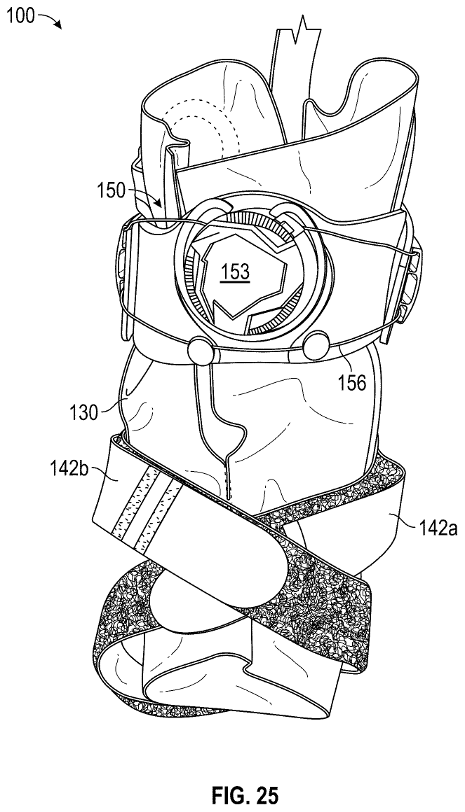

Accordingly, FIGS. 25-29 illustrate yet another embodiment of a short brace 100, in accordance with some example embodiments. In comparison to brace 100 of FIG. 1-4, brace 100 of FIG. 25-29, essentially, has a dual strap figure-8-type arrangement utilizing a similar first strap 142a and corresponding fastener 144a, and then adding another, second strap 142b and corresponding fastener 144b having a mirror fixation, orientation and application to the first strap 142a and fastener 144a. As previously described, fasteners 144a, 144b may comprise rivet and ring arrangements, such as d-rings, plastic chafes and/or fabric chafes.

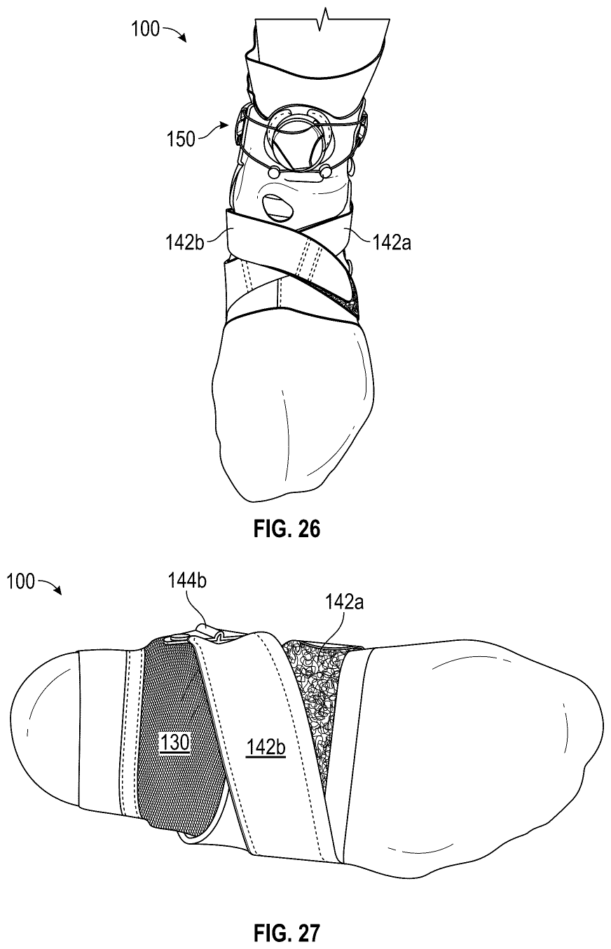

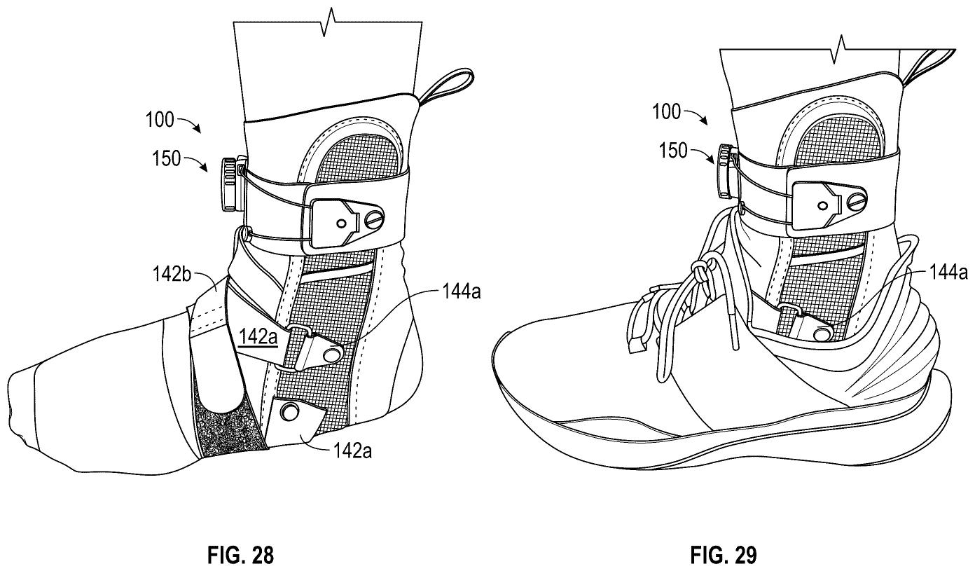

Specifically, FIG. 25 illustrates a front view of such a brace 100. FIG. 26 illustrates a front elevated view of brace 100 of FIG. 25 disposed on a wearer's foot. FIG. 27 illustrates a bottom view of brace 100 of FIG. 26. FIG. 28 illustrates a lateral side view of brace 100 of FIG. 26. And FIG. 29 illustrates a lateral side view of brace 100 of FIG. 26 disposed in a shoe of the wearer.

Brace 100 comprises a first midfoot strap 142a and corresponding first fastener 144a. First midfoot strap 142a is configured to wrap at least partially around a midfoot (e.g., the arch area) of the wearer over an outside of fabric portion 130 of brace 100. Both first fastener 144a and a first end of first midfoot strap 142a may be coupled to a lateral side or bottom of fabric portion 130 at positions that are both inferior to or distal of the ankle of the wearer, or to a similar portion of upright assembly 110 disposed therein. Strap 142a may extend from this first fixed end, under the foot of the wearer, up a medial side of the midfoot of the wearer, over a top of the foot of the wearer inferior to or distal of the ankle joint, through a slot or around a bar of first fastener 144a, double back and secure to itself. Accordingly, a second end of first strap 142a may comprise hook and loop fasteners or complementary snap (e.g., snap-button) fasteners to facilitate the above-described self-securing. First fastener 144a may comprise a rivet and ring arrangement, such as a d-ring, plastic chafe and/or fabric chafe coupled to the lateral side of fabric portion 130, or similar position on upright assembly 110 disposed therein, inferior to or distal of the ankle of the wearer.

Brace 100 further comprises a second midfoot strap 142b and corresponding second fastener 144b. Second midfoot strap 142b is also configured to wrap at least partially around a midfoot (e.g., the arch area) of the wearer over an outside of fabric portion 130 of brace 100, but in an opposite orientation compared to first strap 142a. Both second fastener 144b and a first end of second midfoot strap 142b may be coupled to a medial side or bottom of fabric portion 130 at positions that are both inferior to or distal of the ankle of the wearer, or to a similar portion of upright assembly 110 disposed therein. Strap 142b may extend from this first fixed end, under the foot of the wearer, up a lateral side of the midfoot of the wearer, over a top of the foot of the wearer inferior to or distal of the ankle joint, through a slot or around a bar of second fastener 144b, double back and secure to itself. Accordingly, a second end of second strap 142b may comprise hook and loop fasteners or complementary snap (e.g., snap-button) fasteners to facilitate the above-described self-securing. Second fastener 144b may comprise a rivet and ring arrangement, such as a d-ring, plastic chafe and/or fabric chafe coupled to the medial side of fabric portion 130, or similar position on upright assembly 110 disposed therein, inferior to or distal of the ankle of the wearer.

Brace 100 further comprises an adjustable cuff assembly configured to encircle the lower leg of the wearer over fabric portion 130 and apply medial-lateral force to the lower leg of the wearer to, thereby, prevent a tibia and/or fibula of the wearer from separating relative to one another while brace 100 is worn. Any adjustable cuff assembly 150, 160, 170, 180 and/or 190 as described herein is contemplated.

In some embodiments, straps 142a, 142b may have a single, unchanging width along their entire length of extent. In some other embodiments, straps 142a, 142b may have a varying width along at least a portion of their extent. For example, at least portions of one or both of straps 142a, 142b configured to be disposed against the underside of the arch of the foot of the wearer may have a first width (e.g., 2 inches), then tapering to a second width (e.g., 1 inch) that is narrower than the first width nearer the second end that configured to extend through fastener 144a, 144b.

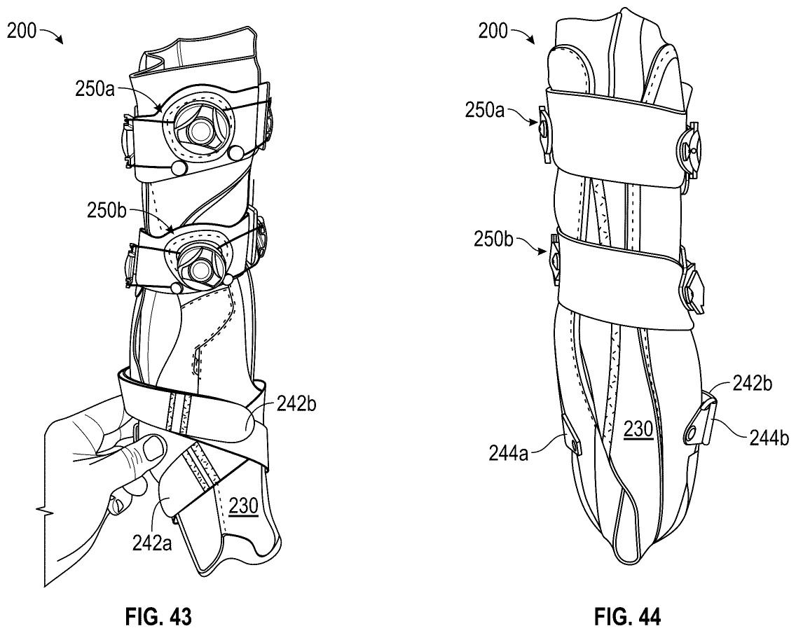

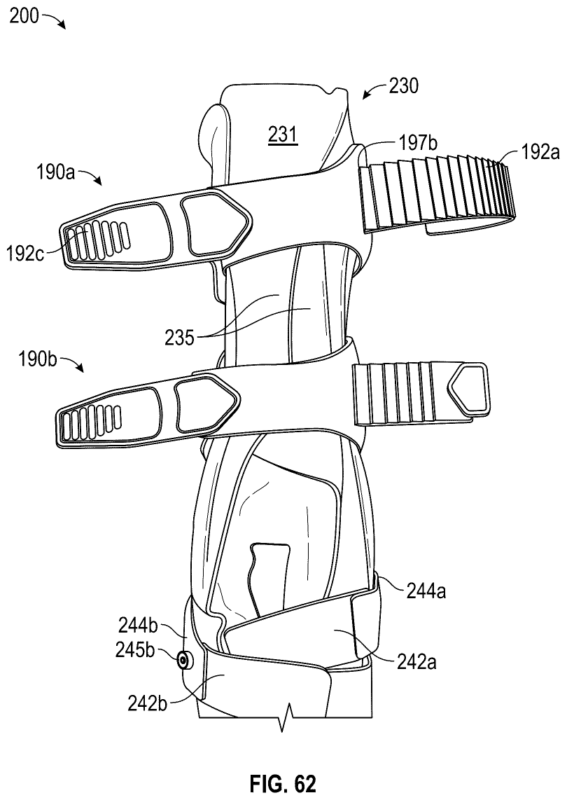

FIGS. 55 and 57-66 illustrate yet other embodiments of braces, in accordance with some example embodiments. Similar to brace 100 of FIG. 25-29 (or braces 200 of FIGS. 43-54), brace 100 of FIGS. 55 and 56-66 has a dual strap figure-8-type arrangement, for example in braces 100, utilizing first strap 142a and corresponding fastener 144a as well as second strap 142b and corresponding fastener 144b having a mirror fixation, orientation and application to first strap 142a and first fastener 144a. As previously described, fasteners 144a, 144b may comprise rivet and ring arrangements, such as d-rings, plastic chafes and/or fabric chafes. However, aspects of brace 100 described in connection with any of FIGS. 55 and 57-61 are also contemplated for taller braces 200 described herein, for example, utilizing first strap 242a and fastener 244a and second strap 242b and fastener 244b, as will be described in more detail in connection with larger brace embodiments 200 below. For example, at least FIGS. 62-66 illustrate embodiments of such a taller brace 200.

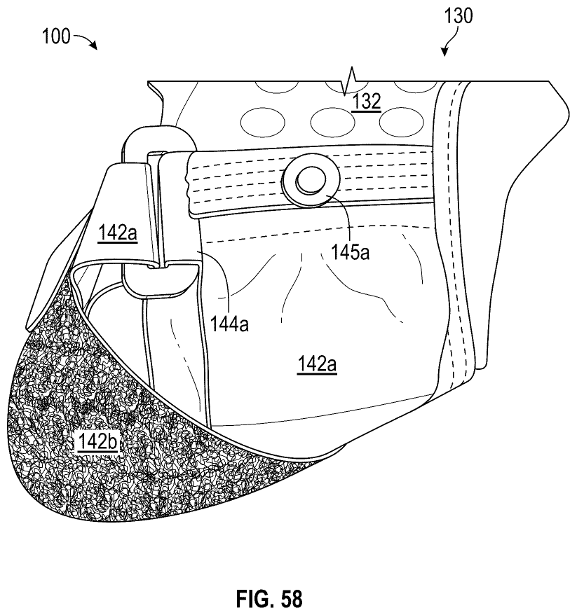

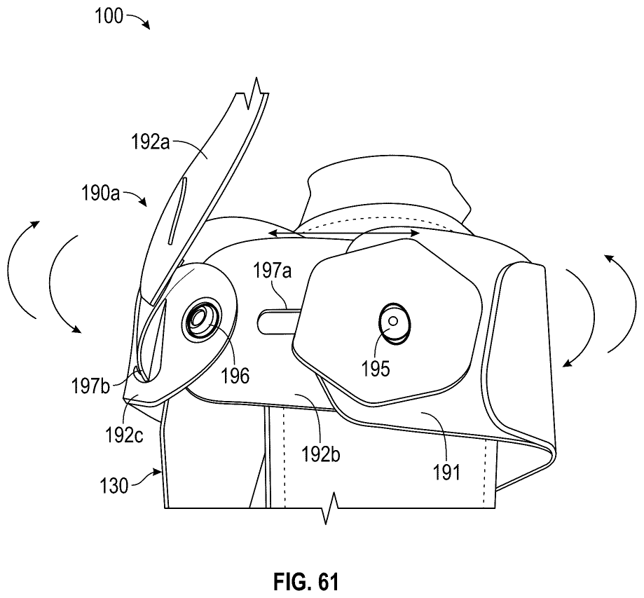

Specifically, FIG. 55 illustrates a front view of such a brace 100 being worn by a user. FIG. 57 illustrates a side view of a portion of several versions of a brace, some including brace 100 of FIG. 55, having varying sizes (e.g., small, medium, large). For example, at least a lower portion of a taller brace 200, as will be otherwise described in connection with one or more figures, e.g., FIGS. 43-54, is also shown in FIG. 57. FIG. 58 illustrates brace(s) 100 of FIG. 55 as viewed from an opposite side from that shown in FIG. 57. Accordingly, any taller brace 200 may also employ such features as shown and described in connection with FIG. 58. FIGS. 60 and 61 illustrate opposite sides of yet another embodiment of a cuff assembly 190, one or more of which is/are utilized by brace 100 of FIGS. 55 and 57-61, or by any other brace 100, 200, short or tall, described anywhere in this disclosure, for example, at least FIGS. 62-66. FIG. 61 illustrates a portion of a cuff assembly 190 (e.g., 190a) disposed around fabric portion 130 of brace 100.

As previously described in connection with at least one embodiment, brace 100 comprises a fabric portion 130 (e.g., a closeable tubular, sleeve as shown in FIG. 5) having a rigid upright assembly 110 (see, e.g., FIG. 6) disposed, or sewn, at least partially therein. For example, FIG. 5 illustrates fabric portion 130 configured to receive the lower leg of the wearer into fabric portion 130 from an anterior thereof. As shown, fabric portion 130 may comprise a stretchy portion 131 (e.g., a first portion having a desired amount of stretchability, e.g., a material and/or fabric configured to stretch, such as nylon) comprising hook and loop front closure flaps 135 configured to receive the lower leg of the wearer therein through the anterior of fabric portion 130 when flaps 135 are open. In some embodiments, stretchy portion 131 comprises substantially an entirety of the base layer of fabric portion 130. Flaps 135 are then configured to couple to one another, thereby enclosing the lower leg of the wearer in fabric portion 130. When so secured, fabric portion 130 comprises a foot hole 137 at the distal end thereof, a lower leg hole 138 at the proximal end thereof, and a heel hole 136 in a posterior portion thereof, between foot hole 137 and lower leg hole 138. Fabric portion 130 further comprises a less stretchy portion 132 (e.g., a second portion having a lower degree of stretchability compared to the first, stretchy portion 131, e.g., in some embodiments, a material and/or fabric configured to substantively not stretch such as teryene mesh), disposed on medial and lateral sides thereof. Less stretchy portion 132 is configured to extend along the underside and both the lateral and medial sides of fabric portion 130 and encircle or encase at least a portion of upright assembly 110 therein and/or between portions 131 and 132 (see, e.g., FIGS. 5 and 6). In some embodiments, less stretchy portion 132 comprises an edge finisher (e.g., teryene grograin binding 133) that is, then, sewn to stretchy portion 131. In some embodiments, a posterior spine (e.g., from heel hole 136 to the posterior upper (proximal) edge) of fabric portion 130 may comprise a polypropylene webbing 134 for added comfort and/or stretch compliance along the Achilles tendon of the wearer. In some embodiments, webbing 134 may comprise a seam tape. In some embodiments, webbing 134 may comprise a material with a relatively increased ability to stretch compared to polypropylene, such as polyspandex, or in some such embodiments, a polyspandex seam tape. In some embodiments, fabric portion 130 comprises a finger loop (e.g., a polypropylene finger loop) configured to aid the wearer's donning of brace 100.

Brace 100 comprises a first midfoot strap 142a and corresponding first fastener 144a. First midfoot strap 142a is configured to wrap at least partially around a midfoot (e.g., the arch area) of the wearer over an outside of fabric portion 130 of brace 100. In contrast to several other embodiments, where fastener 144a and midfoot strap 142a are coupled to fabric portion 130 and/or upright assembly 110 at different locations, see, e.g., FIG. 57, in the embodiments shown in at least FIGS. 55 and 57-61, both first fastener 144a and a first end of first midfoot strap 142a may be coupled to a lateral side or bottom of fabric portion 130 at a same position, for example, utilizing a single rivet, or rivet and washer combination, 145a (see, e.g., FIG. 58) that is inferior to or distal of the ankle of the wearer while properly disposed in brace 100, or to a similar portion of upright assembly 110 disposed therein. Such embodiments consolidate the fastening of fastener 144a and strap 142a to a single location and fastening feature. For example, as shown in FIG. 58, strap 142a and the fabric chafe holding the D-ring or plastic chafe, may be sewn together into a single seam and/or fabric feature 144a, through which fastener (e.g., rivet, rivet/washer) 145a passes and secures to fabric portion 130 and/or upright assembly 110 therein. This reduces part count and ensures the securing force of the fastener 144a and strap 142a arrangement is directed toward or away from a same location on brace 100 that is inferior to or distal of the ankle of the wearer (e.g., single rivet, or rivet and washer combination, 145a). This advantageously improves consistency and effectiveness of the tension in strap 142a at securing the ankle of the user without the strap bunching, gapping and/or over-tensioning when the user's foot dorsiflexes and/or plantarflexes within brace 100.

Strap 142a may extend from this first fixed end, under the foot of the wearer, up a medial side of the midfoot of the wearer, over a top of the foot of the wearer inferior to distal of the ankle joint, through a slot or around a bar of first fastener 144a, double back and secure to itself. Accordingly, a second end of first strap 142a may comprise hook and loop fasteners or complementary snap (e.g., snap-button) fasteners to facilitate the above-described self-securing. First fastener 144a may comprise a rivet and ring arrangement, such as a d-ring, plastic chafe and/or fabric chafe, coupled to the lateral side of fabric portion 130, or similar position on upright assembly 110 disposed therein, inferior to or distal of the ankle of the wearer.

Brace 100 further comprises a second midfoot strap 142b and corresponding second fastener 144b, which are secured as first midfoot strap 142a and corresponding first fastener 144a but to an opposite (medial/lateral) side of fabric portion 130 and/or upright assembly 110 disposed therein. Second midfoot strap 142b is also configured to wrap at least partially around a midfoot (e.g., the arch area) of the wearer over an outside of fabric portion 130 of brace 100, but in an opposite orientation compared to first strap 142a. Both second fastener 144b and a first end of second midfoot strap 142b may be coupled to a medial side or bottom of fabric portion 130 at a same position, for example, utilizing a single rivet, or rivet and washer combination, 145b (see, e.g., FIG. 57) that is inferior to or distal of the ankle of the wearer, and/or to a similar portion of upright assembly 110 disposed therein. For example, as shown in FIG. 57 and similar to that shown in FIG. 58, strap 142b and the fabric chafe holding the D-ring or plastic chafe, may be sewn together into a single seam and/or fabric feature, through which fastener (e.g., rivet, rivet/washer) 145b passes and secures to fabric portion 130 and/or upright assembly 110 therein. Such embodiments consolidate the fastening of fastener 144b and strap 142b to a single location and fastening feature. This reduces part count and ensures the securing force of the fastener 144b and strap 142b arrangement is directed toward or away from a same location on brace 100 that is inferior to or distal of the ankle of the wearer (e.g., single rivet, or rivet and washer combination, 145b). This advantageously improves effectiveness of maintaining a consistent tension in strap 142b so as to secure the ankle of the user without the strap bunching, gapping and/or over-tensioning when the user's foot dorsiflexes and/or plantarflexes within brace 100.

Strap 142b may extend from this first fixed end, under the foot of the wearer, up a lateral side of the midfoot of the wearer, over a top of the foot of the wearer inferior to or distal of the ankle joint, through a slot or around a bar of second fastener 144b, double back and secure to itself. Accordingly, a second end of second strap 142b may comprise hook and loop fasteners or complementary snap (e.g., snap-button) fasteners to facilitate the above-described self-securing. Second fastener 144b may comprise a rivet and ring arrangement, such as a d-ring, plastic chafe and/or fabric chafe, coupled to the medial side of fabric portion 130, or similar position on upright assembly 110 disposed therein, inferior to or distal of the ankle of the wearer.

In some embodiments, straps 142a, 142b may have a single, unchanging width along their entire length of extent. In some other embodiments, straps 142a, 142b may have a varying width along at least a portion of their extent. For example, at least portions of one or both of straps 142a, 142b configured to be disposed against the underside of the arch of the foot of the wearer may have a first width (e.g., 2 inches), then tapering to a second width (e.g., 1 inch) that is narrower than the first width nearer the second end that configured to extend through fastener 144a, 144b.

Brace 100 further comprises one or more adjustable cuff assemblies 190. For example, in some embodiments, brace 100 comprises a single adjustable cuff assembly (e.g., 190a or 190b as would be shown in FIG. 55 with 190b or 190a omitted). However, the present disclosure is not so limited and brace 100 may alternatively comprise first and second adjustable cuff assemblies (e.g., 190a, 190b as shown in FIG. 55) configured to encircle the lower leg of the wearer over fabric portion 130 and apply medial-lateral force to the lower leg of the wearer to, thereby, prevent a tibia and/or fibula of the wearer from separating relative to one another while brace 100 is worn. While adjustable cuff assembly 150, 160, 170, 180, 190 as described anywhere herein is contemplated, embodiments according to at least FIGS. 55 and 57-66 include at least one of cuff assemblies 190a and/or 190b having pivoting and slotted ratchet style cuffs, as described below and/or anywhere in this disclosure. For example, embodiments of brace 200 utilizing cuff assemblies 190a and 190b is shown in at least FIGS. 62-66 and may have substantially similar and/or identical construction to that of brace 100 except as explicitly described differently.

Cuff assembly 190 comprises a posterior cuff portion 191. A first anterior cuff portion 192a is pivotally or rotatably coupled to a first end or portion of posterior cuff portion 191 about an axle, rivet or similar feature 194. At least one side or surface of first anterior cuff portion 192a comprises a plurality of ratcheting features 198a, for example, a series of protrusions, e.g., angular protrusions or teeth, configured to be received by a complementary ratcheting and/or locking feature of assembly 190.

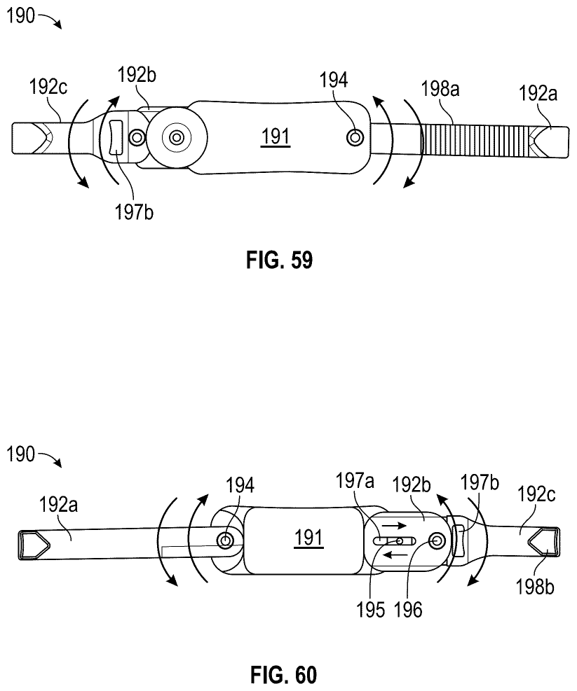

A second anterior cuff portion 192b is slidingly or translatably coupled to a second end or portion of posterior cuff portion 191, opposite the first portion to which first anterior cuff portion 192a is pivotally or rotatably coupled. Specifically, second anterior cuff portion 192b may comprise a slot 197a configured to slidingly receive a post 195 coupled to the second end or portion of posterior cuff portion 191. However, the present disclosure is not so limited, and slot 197a could alternatively be disposed in the second end or portion of posterior cuff portion 191 and post 195 coupled to second anterior cuff portion 192b. Slot 197a may extend in a horizontal direction as shown in FIGS. 59 and 60, for example, extending substantially parallel to a length of circumferential extension of posterior cuff portion 191 around a lower leg of the wearer of brace 100. Accordingly, second anterior cuff portion 192b is configured to slidingly translate with respect to posterior cuff portion 191 within a range of translation defined by post 195 being maximally disposed to one end within slot 197a and post 195 being maximally disposed to the other end within slot 197a. Slot 197a is disposed toward a first end or portion of second anterior cuff portion 192b (e.g., an opposite end of second anterior cuff portion 192b from the end pivotally coupled to a third anterior cuff portion 192c below).

A third anterior cuff portion 192c is pivotally or rotatably coupled to a second end or portion of second anterior cuff portion 192b, opposite of the first end or portion, about an axle, rivet or similar feature 196. Third anterior cuff portion 192c further comprises a slot or aperture 197b configured to receive at least a portion of first anterior cuff portion 192a therethrough. For example, the portion of first anterior cuff portion 192a comprising ratcheting features 198a may be configured to extend through slot 197b. In some embodiments, the ratcheting action occurs between features 198a and the perimeter of aperture 197b. For example, aperture 197b may have a width slightly greater than a width of at least a portion of first cuff portion 192a configured to extend through aperture 197b of third anterior cuff portion 192c, so as to allow the pulling of first cuff portion 192a through aperture 197b and, so, the tightening of cuff assembly 190 (e.g., 190a and/or 190b). However, aperture 197b may also have a height (e.g., substantially perpendicular to the above-described width) that is slightly smaller than a greatest height of ratcheting features 198a of first anterior cuff portion 192a, such that, when first cuff portion 192a is pulled through aperture 197b, ratcheting features 198a deform slightly so as to fit through aperture 197b and then spring back to their original (e.g., unloaded) profile, so as to hold first cuff portion 192a in a tightened orientation with respect to third anterior cuff portion 192c.Once extended through aperture 197b, terminal ends of first anterior cuff portion 192a and/or third anterior cuff portion 192c may comprise one of complementary hook or loop fasteners configured to secure to the other of the complementary loop or hook feature on the side of the brace. For example, an outward-facing surface of any portion of cuff assembly 190 (e.g., 190a, 190b) may comprise such a complementary hook and/or loop fastener surface. For example, posterior cuff portion 191, or a pad disposed on posterior cuff portion 191 (e.g., as shown disposed around and/or over post 195 in FIG. 61) may comprise such a complementary hook and/or loop fastener surface. However, the present disclosure is not so limited, and any other suitable fastening means for first anterior cuff portion 192a and/or third anterior cuff portion 192c are also contemplated.

Accordingly, in operation, cuff assembly 190 is configured to be adjustable in multiple dimensions simultaneously. For example, translation of post 195 in slot 197a allows adjustment of the depth (e.g., anterior-posterior dimension) of cuff assembly 190. In some embodiments, post 195 and slot 197a may be fixed and/or otherwise locked in a desired orientation with respect to one another, thereby fixing and/or otherwise locking posterior cuff portion 191 with respect to second anterior cuff portion 192b. Moreover, the use of axles, rivets or similar features 194, 196 allow anterior cuff portions 192a and 192c to pivot with respect to posterior cuff portion 191 and/or second anterior portion 192b, respectively. When cuff assembly 190 is tightened, posterior cuff portion 191 is disposed against posterior and side portions of the lower leg of the wearer, above the ankle joint, and first, second and third anterior cuff portions 192a, 192b, 192c are disposed against side and anterior portions of the lower leg of the wearer, above the ankle joint. In some embodiments, padding is provided on an inside surface of one or more of posterior cuff portion 191 and first, second and/or third anterior cuff portions 192a, 192b, 192c. In some embodiments, padding is provided on an outside surface of one or more of posterior cuff portion 191 and first, second and/or third anterior cuff portions 192a, 192b, 192c. As can be seen in FIG. 55, brace 100 may comprise a first cuff assembly 190 (e.g., 190a) disposed over or around fabric portion 130 adjacent the superior or proximal edge of fabric portion 130 and superior to, or proximal of, the ankle of the wearer. While brace 100 may comprise a single cuff assembly 190, in some embodiments, brace 100 may also comprise a second cuff assembly 190b over or around fabric portion 130 just superior to, or proximal of, the ankle of the wearer. In some such embodiments, second cuff assembly 190b is disposed between first cuff assembly 190a and the ankle of the wearer. Accordingly, in operation, donning brace 100, and adjusting one or more of posterior cuff portion 191, and/or anterior cuff portions 192a, 192b, 192c to the desired orientation(s) around the lower leg of the user, and tightening cuff assemblies 190a, 190b pulls anterior cuff portions 192a, 192b, 192c and posterior cuff portion 191 together and applies the desired amount of medial-lateral force to the lower leg of the wearer to prevent the tibia and/or fibula of the wearer from separating relative to one another while brace 100 is worn. However, the present disclosure is not so limited. And brace 100 utilizing only a single cuff assembly 190 may be configured to dispose that single cuff assembly 190 in either the lower location described above for second cuff assembly 190b or the upper location described above for first cuff assembly 190a.

In some embodiments, brace 100 may, optionally, comprise a separate foot plate 120, for example as shown in FIG. 4. Foot plate 120 has a foot-facing surface shaped complementary to the underside of the arch of the foot of the wearer to, thereby, mate with and provide arch support to the arch of the wearer. In some embodiments, foot plate 120 comprises plastic and/or an elastomeric material. In some embodiments, foot plate 120 is heat formable, for example, becoming sufficiently malleable within a temperature range of 150-300° Fahrenheit to form, or deform, to a desired shape complementary to at least a portion of the underside of the wearer's foot. In some embodiments, foot plate 120 may be heat-formed by application of an exothermic heat patch and/or by subjecting foot plate 120 to heat from a heat gun. In some embodiments, foot plate 120 is configured to be disposed between the foot of the wearer and at least part of an inner surface of fabric portion 130. For example, when optional foot plate 120 is properly inserted into brace 100 and brace 100 is properly worn by the wearer, foot plate 120 may be held fast against the underside of the arch of the foot of the wearer by at least fabric portion 130. In some embodiments, at least an anterior end of foot plate 120 may extend anteriorly beyond an anterior-most extent of an under-foot portion of fabric portion 130. In some embodiments, at least a posterior end of foot plate 120 may extend posteriorly beyond a posterior-most extent of an under-foot portion of fabric portion 130, for example, posteriorly beyond an anterior edge of the heel hole of fabric portion 130 configured to expose the heel of the wearer therethrough.

### Large Brace Embodiments

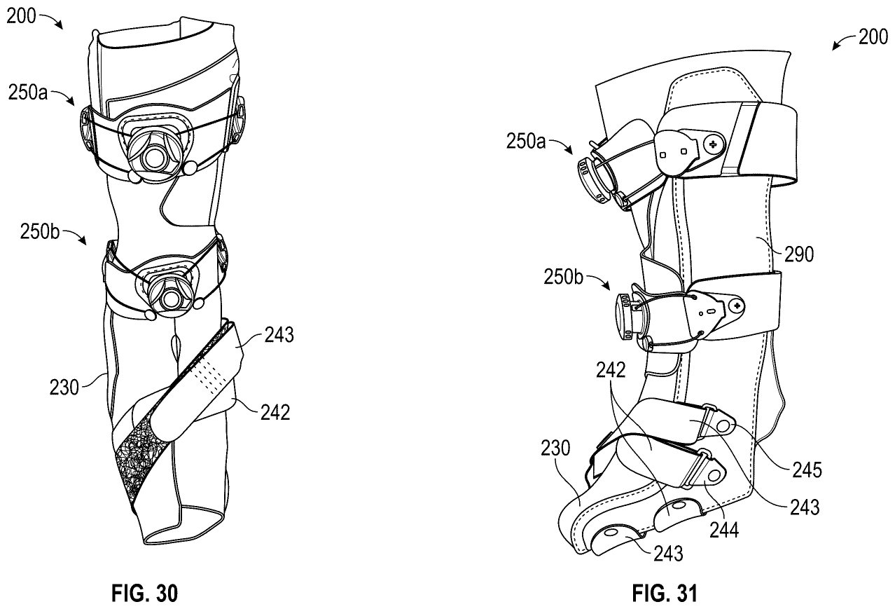

FIGS. 30-54 and at least FIGS. 57, 59-60 and 62-66 illustrate different views of one or more embodiments of a taller ankle brace 200, in accordance with some embodiments. While not limited to such uses, such tall braces 200 may be utilized to treat high ankle sprains. Such tall braces 200 may be configured to affect high ankle biomechanics including but not limited to preventing or substantially reducing foot eversion, tibia-fibula separation, foot pronation, foot dorsiflexion, and/or to provide a desired amount of forefoot abduction. Due to the balanced nature and/or design of brace 200, brace 200 also has secondary limiting and/or supportive effects regarding plantarflexion and supination. Brace 200 according to one or more of FIGS. 30-54 and at least FIGS. 57, 59-60 and 62-66 includes one or more of: a hybrid embedded frame having an unarticulated lateral side with a jointed medial side configured to create a torsional affect with ankle motion during ambulation; double, dual, or inferior & superior medial/lateral cuffs 250a, 250b (or inferior and/or medial/lateral cuffs similar to or the same as 190a, 190b shown in FIGS. 55 and 59-66) configured to prevent a tibia and/or fibula of the wearer from separating relative to one another while brace 200 is worn; a midfoot strap 242 and associated fastener 244; a forefoot abduction strap 243 and associated fastener 245, double midfoot straps (or “figure 8” straps) 242a, 242b and associated fasteners 244a, 244b configured to, simultaneously, be secured below an ankle joint of the wearer and allow dorsiflexion and/or plantarflexion without affecting tension in the strap(s) 242a, 242b; and/or a unique lateral boarder design. In some embodiments, the boarder design comprises the lateral padded boarder and a medial padded boarder with limited or “frameless” plantar surface(s).

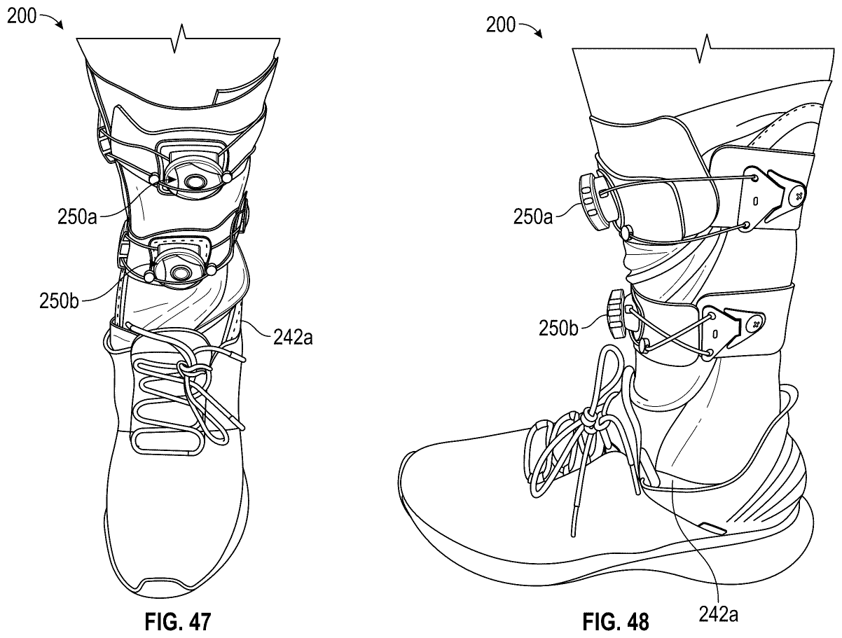

While several taller ankle braces 200, described herein, are illustrated as having cuff assemblies comprising reel-and-lace features, the present disclosure is not so limited, and also contemplates tall braces 200 as otherwise described anywhere, herein, alternatively including cuff assemblies 190 (e.g., first and/or second cuff assemblies 190a, 190b) as described, for example, in connection with FIGS. 55 and 47-66.

Moreover, while several taller ankle braces 200, described herein, are illustrated as having first and second straps 242a, 242b and first and second fasteners 244a, 244b attached at different positions on brace 200, the present disclosure is not so limited, and the present disclosure also contemplates tall braces 200 as otherwise described anywhere herein, alternatively having first strap 242a and corresponding fastener 244a being coupled to the same position on fabric portion 230 and/or upright assembly 210 utilizing a single rivet, or rivet and washer combination, 245a (see, e.g., FIG. 58) that is inferior to or distal of the ankle of the wearer, or to a similar portion of upright assembly 210 disposed therein. Similarly, second strap 242b and corresponding fastener 244b may be coupled to the same position on fabric portion 230 and/or upright assembly 210 utilizing a single rivet, or rivet and washer combination, 245b (see, e.g., FIG. 57) that is inferior to or distal of the ankle of the wearer, or to a similar portion of upright assembly 210 disposed therein.

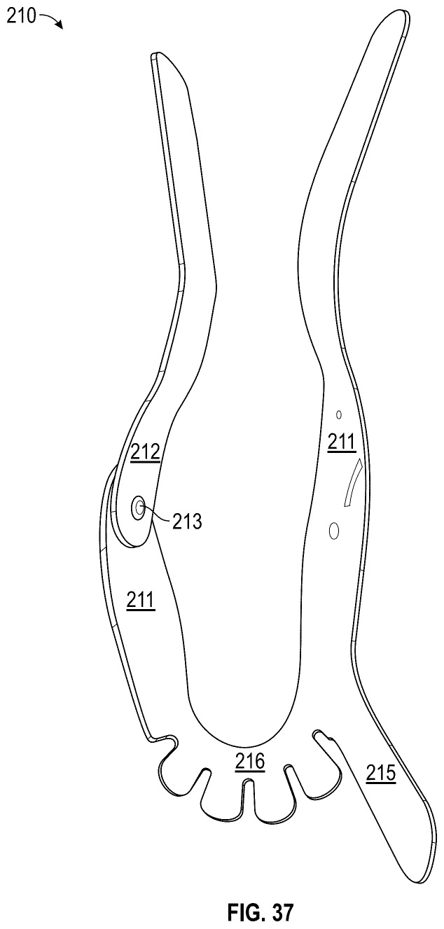

In some embodiments, a rigid or semi-rigid upright assembly 210 (see, e.g., FIG. 37 or FIGS. 38-42) may be embedded in fabric portion 230 (e.g., soft goods) to, thereby, simultaneously provide a rigid frame and frame cover that also provides a soft interface with the lower leg of the wear. Accordingly, fabric portion 230 may also be configured to house at least a portion of such an upright assembly therein.

In some embodiments, for example as shown in FIG. 37, the lateral upright is not articulated, while the medial side is articulated (or vice versa, as also contemplated by this disclosure). Such a configuration is designed to create a diagnosis-specific torsional affect opposing or resisting external rotation of the ankle joint (e.g., opposing ankle rotation in the direction that caused or exacerbated the ankle injury) during ambulation. For example, when the wearer dorsiflexes the frame bends in a manner that resists external rotation. And, in such configurations, the lateral lower upright extends up the lateral side of the lower leg of the wearer without a jointed upper upright, while the medial side of the upright assembly is as described in the immediately preceding paragraph.

Where neither the lateral or medial upright is articulated, both lateral and medial lower uprights extend up the respective lateral or medial side of the lower leg of the wearer without coupling to respective jointed or rotatable upper uprights. In any embodiment of brace 200, such an upright assembly may be sufficiently rigid to provide support to the lower leg of the wearer, comprising, for example and not limitation aluminum, stainless steel, carbon fiber, a rigid plastic, or another composite construction.

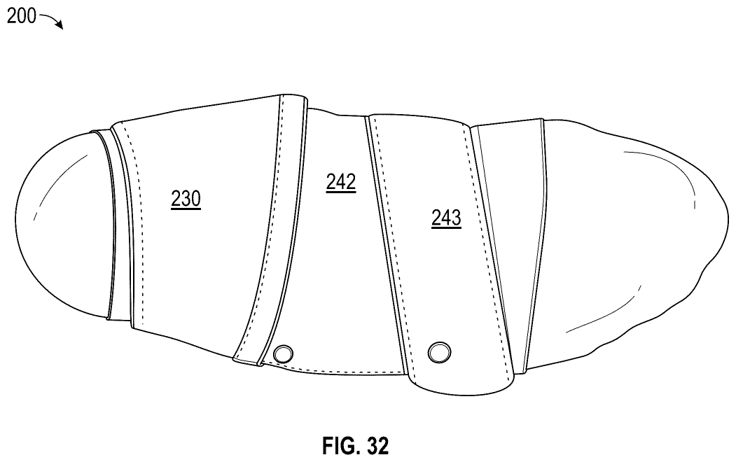

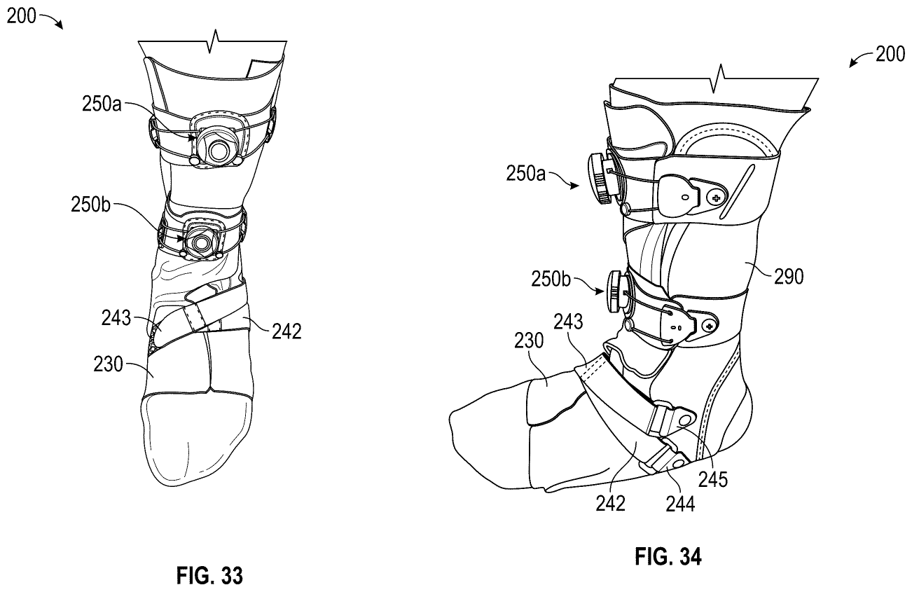

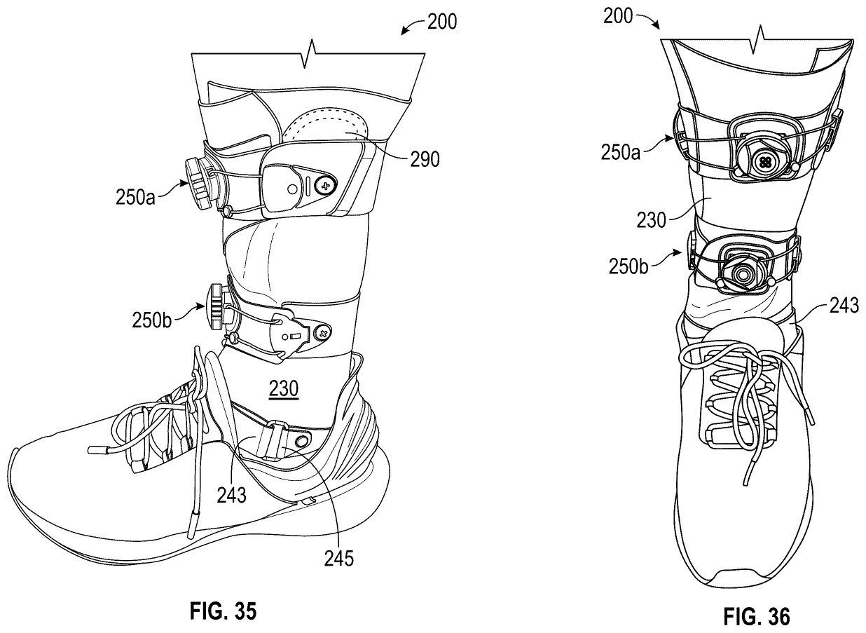

FIGS. 30-36 illustrate an embodiment of a tall ankle brace 200. For example, FIG. 30 illustrates a front elevated view of such a brace 200. FIG. 31 illustrates a side view of brace 200 of FIG. 30. FIG. 32 illustrates a bottom view of brace 200 of FIG. 30. FIG. 33 illustrates yet another elevated front view of brace 200 of FIG. 30. FIG. 34 illustrates yet another side view of brace 200 of FIG. 30. FIG. 35 illustrates a side view of brace 200 of FIG. 30 disposed on a wearer's lower leg and the wearer's braced foot inserted in a shoe. FIG. 36 illustrates an elevated front view of brace 200 of FIG. 30 disposed on a wearer's lower leg and the wearer's braced foot inserted in a shoe.

As shown, brace 200 comprises a fabric portion 230 (e.g., a closeable, tubular sleeve) having a rigid upright assembly 210 disposed, or sewn, at least partially therein. For example, similar to that shown in FIG. 5 with respect to fabric portion 130 of the short braces 100, fabric portion 230 is configured to receive the lower leg of the wearer into fabric portion 230 from an anterior thereof. Fabric portion 230 may comprise a stretchy portion (e.g., a first portion comprising a material and/or fabric having a desired amount of stretchability, such as nylon such as stretchy portion 131 of FIG. 5) comprising a hook and loop front closure flaps (see, e.g., closure flaps stretchy portion 135 of FIG. 5) configured to receive the lower leg of the wearer therein through the anterior of fabric portion 230 when the flaps are open. In some embodiments, stretchy portion 231 comprises substantially an entirety of the base layer of fabric portion 230. The flaps are then configured to couple to one another, thereby enclosing the lower leg of the wearer in fabric portion 230. When so secured, fabric portion 230 comprises a foot hole at the distal end thereof, a lower leg hole at the proximal end thereof, and a heel hole (see, e.g., heel hole 136 of FIG. 5) in a posterior portion thereof, between the foot hole and the lower leg hole. Fabric portion 230 further comprises a less stretchy portion (see, e.g., a material and/or fabric having a lower degree of stretchability compared to the first, stretchy portion, such as teryene mesh as for less stretchy portion 132 of FIG. 5) disposed on medial and lateral sides thereof. This non-stretchy portion is configured to extend along the underside and both the lateral and medial sides of fabric portion 230 and encircle or encase at least a portion of an upright assembly 210 disposed therein and/or between portions 231 and 232 (see, e.g., FIGS. 37-42 below). In some embodiments, the less stretchy portion comprises an edge finisher (see, e.g., teryene grograin binding 133) that is, then, sewn to the stretchy portion. In some embodiments, a posterior spine (e.g., from the heel hole to the posterior upper (proximal) edge) of fabric portion 230 may comprise a polypropylene webbing (see, e.g., 134 of FIG. 5) for added comfort and/or stretch compliance along the Achilles tendon of the wearer. As stated above regarding fabric portion 130, in some embodiments, the webbing may comprise a seam tape. In some embodiments, the webbing may comprise a material with a relatively increased ability to stretch compared to polypropylene, such as polyspandex, or in some such embodiments, a polyspandex seam tape. In some embodiments, fabric portion 230 comprises a finger loop (e.g., a polypropylene finger loop) configured to aid the wearer's donning of brace 200.

As also shown in FIGS. 30-36, brace 200 comprises a lateral boarder 290 fixedly coupled to, or integrated as a portion of, a lateral outside surface of fabric portion 230. In some embodiments, lateral boarder 290 is padded to provide extra cushion and bump protection to the lower leg of the wearer. In some embodiments, lateral boarder 290 extends along substantially an entirety of a lateral side of fabric portion 230 and is configured to resist forefoot abduction. Accordingly, lateral boarder 290 is configured to extend along at least the heads of the metatarsals of the wearers foot, a lateral midfoot, a lateral heel and ankle portion, and a lateral side of the lower leg substantially to the top of fabric portion 230 (e.g., mid-calf of the wearer). In some embodiments, lateral boarder 290 is angled anteriorly to maintain an open heel design and to prevent bunching and to aid in shoe fit thereover. In some embodiments, at least a portion of the lateral side of the upright assembly may be disposed within or underneath (toward the leg of the wearer from) lateral boarder 290.

FIG. 37 illustrates an embodiment of an asymmetrically-jointed upright assembly 210 of brace 200 as mentioned above. At least a portion of upright assembly 210 is sewn into fabric portion 230 similar to how upright assembly 110 is sewn into fabric portion 130 for brace 100. Upright assembly 210 comprises an underfoot portion 216, which bends and then extends from both ends, substantially perpendicularly of underfoot portion 216, to form lateral and medial lower uprights 211. In some embodiments, underfoot portion 216 and lateral and medial uprights 211 are monolithic, formed as a single piece that may be bent or otherwise formed into a substantially “U” shaped form, thereby decreasing part count of brace 200. In some embodiments, underfoot portion 216 is formed having a plurality of tabs with material portions of the single piece between missing, removed or molded to so as to form individual tabs that allow the monolithic portion comprising underfoot portion 216 and lateral and medial uprights 211 to bend into the above-described “U”-shaped form. A distal end of a medial upper upright 212 is rotatably or pivotally coupled to a proximal end of the medial lower upright 211 via joint 213, which may comprise an axle, rivet or similar feature. By contrast, the lateral upright 211 extends, unjointed, to a similar height as the proximal end of medial upper upright 212. Upright assembly 210 also comprises an extension 215 protruding from the base of lateral upright 211 in a direction of the toes of the wearer's foot (e.g., substantially perpendicularly to both the medial-lateral extension of underfoot portion 216 and the proximal-distal extension of lateral upright 211. Extension 215 may provide resistance to migration or pivoting of brace 200, or of upright assembly 210 disposed therein, in the anterior direction. As with assembly 110, upright assembly 210 and/or its components may be sufficiently rigid to provide support to the lower leg of the wearer, comprising, for example and not limitation, aluminum, stainless steel, carbon fiber, a rigid plastic, or another composite construction.

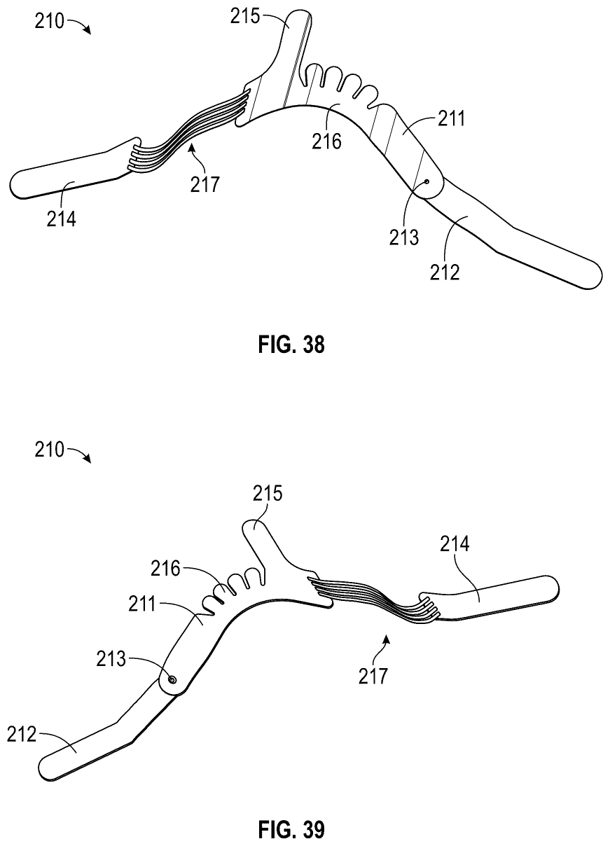

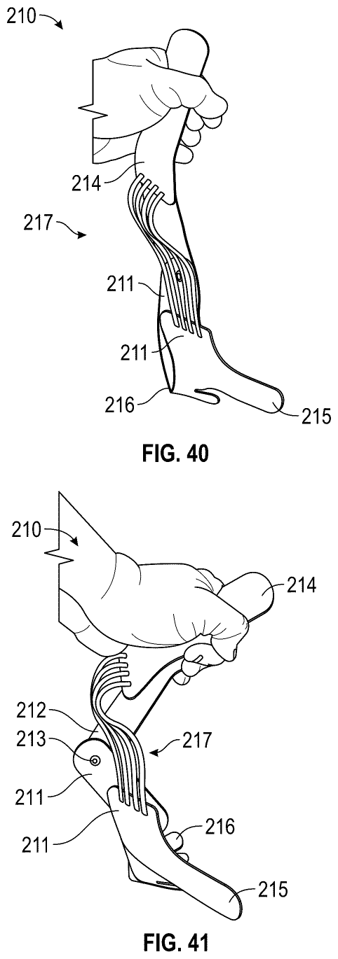

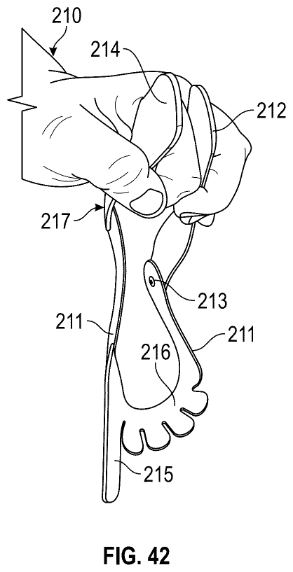

However, the present disclosure is not so limited and other embodiments of an upright assembly 210 are also contemplated. For example, FIGS. 38-42 illustrate an embodiment in which the lateral and medial sides of upright assembly 210 are both articulated, though utilizing different mechanisms. Specifically, FIG. 38 illustrates such an upright assembly 210. FIG. 39 illustrates the upright assembly of FIG. 38 substantially flipped along the horizontal compared to FIG. 38. FIG. 40 illustrates a perspective view of the upright assembly of FIG. 39 in a substantially unbent orientation. FIG. 41 illustrates a perspective view of the upright assembly of FIG. 39 in a substantially bent orientation. And FIG. 42 illustrates a substantially frontal view of the upright assembly of FIG. 41 in the substantially bent orientation.

Here, upright assembly 210 has identical underfoot portion 216, medial and lateral lower uprights 211, pivot 213, medial upper upright 212, and extension 215 as in FIG. 37. However, rather than the lateral side of lower upright 211 extending all the way up to a level of the proximal end of medial upper upright 212, a lateral upper upright 214 is flexibly coupled to lateral lower upright 211 utilizing a multi-finger pivot structure 217. Specifically, pivot structure 217 comprises a plurality of thin fingers, each extending between a proximal end of lateral lower upright 211 and a distal end of lateral upper upright 214. In the unbent orientations of upright assembly 210 shown in FIGS. 38-39, each thin finger has a first portion that extends substantially straight away from lower lateral upright 211 (each first portion extends substantially parallel to one another in this orientation of upright assembly 210) and a second portion having a curvature, for example a curvature of a semi-ovoid (partial perimeter of an oval, or an open, rounded but oblong curvature), and extending to couple with lateral upper upright 214. The second portions of these fingers are configured, and have a specifically shaped curvature and to have a first spacing between adjacent second portions in the unbent orientation. Moreover, the specifically shaped curvature of the second portions of each finger is configured such that, when lateral upper upright 214 is bent with respect to lateral lower upright 211, as shown in FIGS. 40-42, the second portion of each of the multiple fingers bends, increasing its curvature and decreasing a spacing between adjacent second portions from the first spacing as upright assembly 210 is increasingly bent. In some embodiments, with sufficient bending, adjacent second portions may touch one another. In some embodiments, each of the second portions remains in the same plane as one another (e.g., the second portions make contact with one another at a distal portion thereof, but stay in the same plane). An example of this planar bent configuration is best illustrated in the front view of FIG. 42. In FIGS. 38-42, four (4) fingers are shown. However, the present disclosure is not so limited and any suitable number of fingers are also contemplated.

Turning back to FIGS. 30-36, brace 200 comprises a midfoot strap 242 configured to wrap at least partially around a midfoot (e.g., the arch area) of the wearer on an outside of fabric portion 230 of brace 200 and a corresponding fastener 244. Both fastener 244 and a first end of midfoot strap 242 may be coupled to a lateral side or bottom of fabric portion 230 at positions that are both inferior to or distal of the ankle of the wearer, or to a similar portion of upright assembly 210 disposed therein, or to a similar portion of lateral boarder 290. Strap 242 may extend from this first fixed end, under the foot of the wearer, up a medial side of the midfoot of the wearer, over a top of the foot of the wearer inferior to or distal of the ankle joint, through a slot or around a bar of fastener 244, double back and secure to itself, thereby, providing adjustable support at the midfoot of the wearer. Accordingly, a second end of strap 242 may comprise hook and loop fasteners or complementary snap (e.g., snap-button) fasteners to facilitate the above-described self-securing. Fastener 244 may comprise a rivet and ring arrangement, such as a d-ring,, plastic chafe and/or fabric chafe coupled to the lateral side of fabric portion 230, or similar position on upright assembly 210 disposed therein, or to a similar portion of lateral boarder 290, inferior to or distal of the ankle of the wearer.

As shown in FIGS. 30-36, brace 200 also comprises a forefoot strap 243 configured to wrap at least partially around a midfoot and forefoot of the wearer on an outside of fabric portion 230 of brace 200 (e.g., the arch area and at least a medial portion of a forefoot distal of the wearer's midfoot). For example, a first end of forefoot strap 243 may be coupled to a lateral side of fabric portion 230, to a lateral portion of the upright assembly disposed at least partially therein, or to a lateral portion of lateral boarder 290. Strap 243 may extend from this first end, under the foot of the wearer, up a medial side of the forefoot of the wearer, over a top of the foot of the wearer distal of the ankle joint, through a slot or around a bar of a fastener 245, double back and secure to itself. Accordingly, a second end of strap 243 may comprise hook and loop fasteners or complementary snap (e.g., snap-button) fasteners to facilitate the above-described self-securing. Fastener 245 may comprise a rivet and ring arrangement, such as a d-ring, plastic chafe and/or fabric chafe, coupled to a lateral side of fabric portion 230, to a lateral portion of upright assembly 210 disposed at least partially therein, or to a lateral portion of lateral boarder 290. Once strap 243 is secured, the foot of the wear may be substantially immobilized in or at a desired degree of abduction.

Brace 200 also comprises a first adjustable cuff assembly 250a and a second adjustable cuff assembly 250b. First adjustable cuff assembly 250a may be substantially identical to adjustable cuff assembly 150 (or any other adjustable cuff assembly, e.g., 160, 170, 180 and/or 190) as previously described in connection with brace(s) 100, except, potentially, having an increased size, as necessary, to be disposed over fabric portion 230 at the calf portion of the lower leg of the wearer, rather than at the narrower location just above the ankle of the wearer. Second adjustable cuff assembly 250b may be substantially identical to adjustable cuff assembly 150 (or any other adjustable cuff assembly, e.g., 160, 170, 180 and/or 190) as previously described in connection with brace(s) 100 and is configured to be disposed over fabric portion 230 just above the ankle of the wearer.

FIGS. 43-48 illustrate yet another embodiment of a tall ankle brace 200. In comparison to brace 200 of FIGS. 30-36, brace 200 of FIG. 43-48 omits forefoot strap 243 and corresponding fastener 245 and, instead, utilizes double midfoot straps 242a, 242b and corresponding fasteners 244a, 244b. In some embodiments only the medial upright of the upright assembly of brace 200 is articulated (e.g., utilizing upright assembly 210 of FIG. 37). However, the present disclosure is not so limited and in some other embodiments, neither the lateral nor medial uprights are articulated (e.g., both being rigidly fixed). And, in yet other embodiments, both of the lateral and medial uprights are articulated (e.g., utilizing upright assembly 210 of FIGS. 38-42).

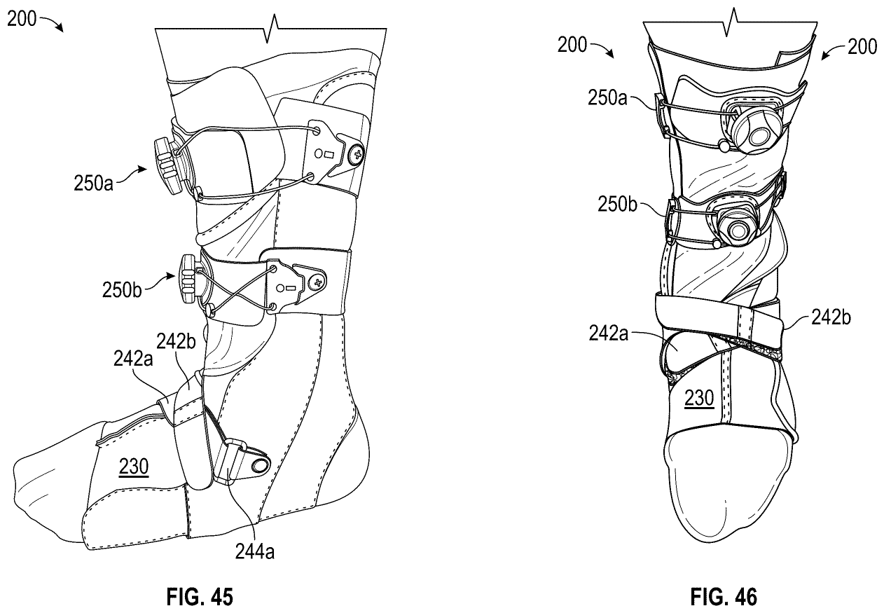

Specifically, FIG. 43 illustrates a front elevated view of brace 200 not disposed on a wearer's lower leg. FIG. 44 illustrates a posterior view of brace 200 of FIG. 43. FIG. 45 illustrates a side view of brace 200 of FIG. 43 disposed on a wearer's lower leg. FIG. 46 illustrates an elevated front view of brace 200 of FIG. 43 disposed on a wearer's lower leg. FIG. 47 illustrates an elevated front view of brace 200 of FIG. 43 disposed on a wearer's lower leg and the wearer's foot disposed in a shoe. And FIG. 48 illustrates a side view of brace 200 of FIG. 43 disposed on a wearer's lower leg and the wearer's foot disposed in a shoe.

Brace 200 comprises a first midfoot strap 242a and corresponding first fastener 244a. First midfoot strap 242a is configured to wrap at least partially around a midfoot (e.g., the arch area) of the wearer over an outside of fabric portion 230 of brace 200. Both first fastener 244a and a first end of first midfoot strap 242a may be coupled to a lateral side or bottom of fabric portion 230 at positions that are both inferior to or distal of the ankle of the wearer, or to a similar portion of upright assembly 210 disposed therein. Strap 242a may extend from this first fixed end, under the foot of the wearer, up a medial side of the midfoot of the wearer, over a top of the foot of the wearer inferior to or distal of the ankle joint, through a slot or around a bar of first fastener 244a, double back and secure to itself. Accordingly, a second end of first strap 242a may comprise hook and loop fasteners or complementary snap (e.g., snap-button) fasteners to facilitate the above-described self-securing. First fastener 244a may comprise a rivet and ring arrangement, such as a d-ring, plastic chafe and/or fabric chafe, coupled to the lateral side of fabric portion 230, or similar position on upright assembly 210 disposed therein, inferior to or distal of the ankle of the wearer.

Brace 200 further comprises a second midfoot strap 242b and corresponding second fastener 244b. Second midfoot strap 242b is also configured to wrap at least partially around a midfoot (e.g., the arch area) of the wearer over an outside of fabric portion 230 of brace 200, but in an opposite orientation compared to first strap 242a. Both second fastener 244b and a first end of second midfoot strap 242b may be coupled to a medial side or bottom of fabric portion 230 at positions that are both inferior to or distal of the ankle of the wearer, or to a similar portion of upright assembly 210 disposed therein. Strap 242b may extend from this first fixed end, under the foot of the wearer, up a lateral side of the midfoot of the wearer, over a top of the foot of the wearer inferior to or distal of the ankle joint, through a slot or around a bar of second fastener 244b, double back and secure to itself. Accordingly, a second end of second strap 242b may comprise hook and loop fasteners or complementary snap (e.g., snap-button) fasteners to facilitate the above-described self-securing. Second fastener 244b may comprise a rivet and ring arrangement, such as a d-ring, plastic chafe and/or fabric chafe, coupled to the medial side of fabric portion 230, or similar position on upright assembly 210 disposed therein, inferior to or distal of the ankle of the wearer.

Brace 200 also comprises a first adjustable cuff assembly 250a and a second adjustable cuff assembly 250b. First adjustable cuff assembly 250a may be substantially identical to adjustable cuff assembly 150 (or any other adjustable cuff assembly, e.g., 160, 170, 180 and/or 190) as previously described in connection with brace(s) 100, except, potentially, having an increased size, as necessary, to be disposed over fabric portion 230 at the calf portion of the lower leg of the wearer, rather than at the narrower location just above the ankle of the wearer. Second adjustable cuff assembly 250b may be substantially identical to adjustable cuff assembly 150 (or any other adjustable cuff assembly, e.g., 160, 170, 180 and/or 190) as previously described in connection with brace(s) 100 and is configured to be disposed over fabric portion 230 just above the ankle of the wearer.

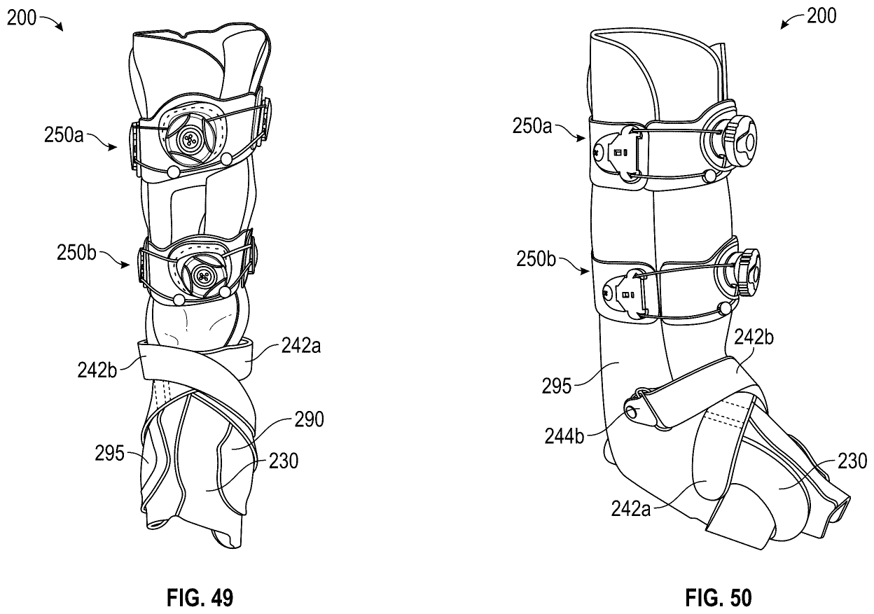

FIGS. 49-54 illustrate yet another embodiment of a brace 200, in accordance with some example embodiments. In comparison to brace 200 of FIGS. 43-48, brace 200 of FIG. 49-54 introduces a medial boarder 295 in addition to lateral boarder 290. While one embodiment contemplates that both the lateral and medial uprights of upright assembly 210 disposed at least partially within fabric portion 230 are articulated (e.g., as previously described in connection with at least FIGS. 38-42), the present disclosure is not so limited and in at least some embodiments, neither the lateral nor medial uprights are articulated, and in at least some other embodiments, only one of the lateral and medial uprights is articulated (e.g., as previously described in connection with FIG. 37).

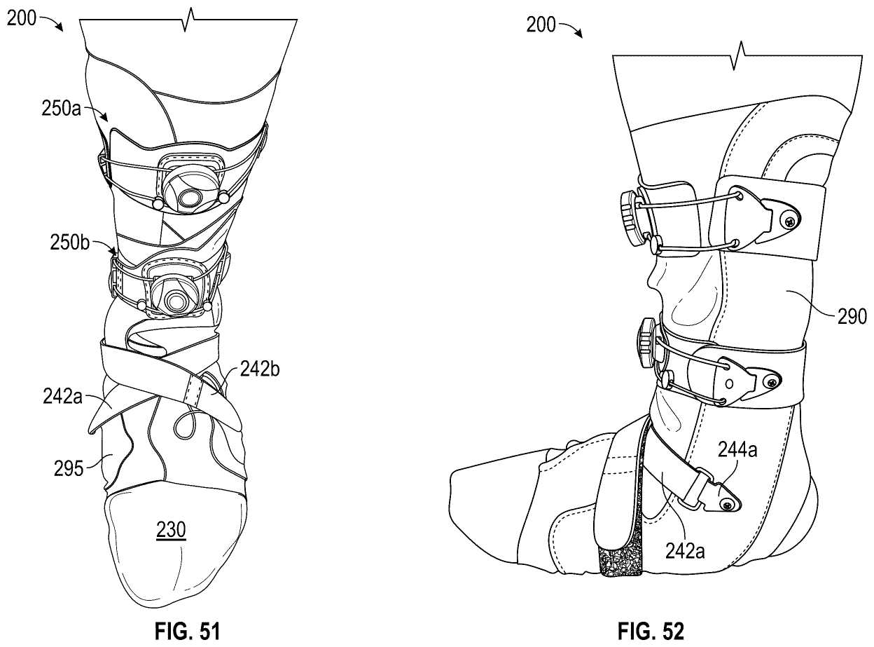

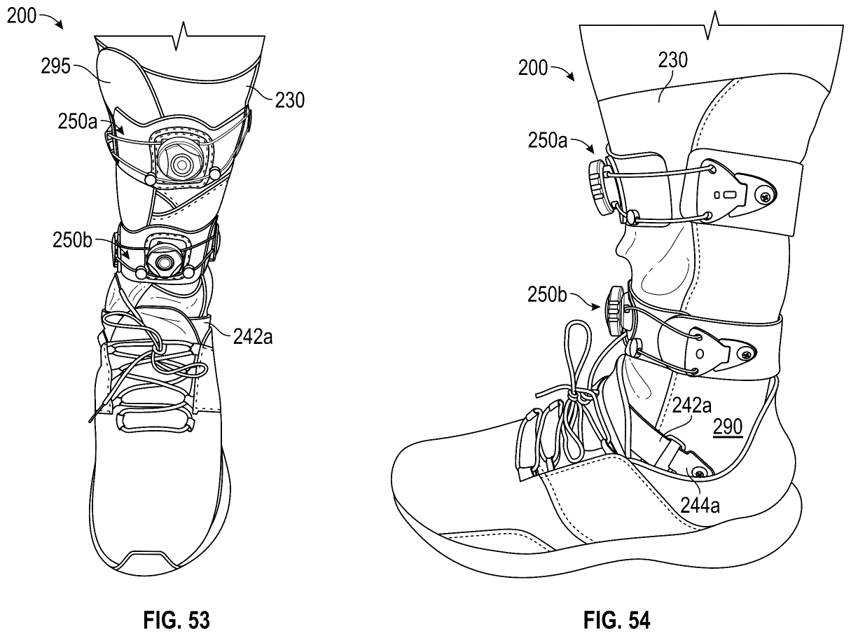

Specifically, FIG. 49 illustrates a front elevated view of brace 200. FIG. 50 illustrates a perspective view of brace 200 of FIG. 49. FIG. 51 illustrates a front elevated view of brace 200 of FIG. 49 disposed on a wearer's lower leg. FIG. 52 illustrates a side view of brace 200 of FIG. 51. FIG. 53 illustrates a front elevated view of brace 200 of FIG. 51 disposed in a shoe. FIG. 54 illustrates a side view of brace 200 of FIG. 43 disposed on a wearer's lower leg without the boarder assembly disposed therearound and the wearer's foot disposed in a shoe.

As shown in FIGS. 49-54, brace 200 comprises first midfoot strap 242a and corresponding first fastener 244a, as well as second midfoot strap 242b and corresponding second fastener 244b as previously described. Brace 200 also comprises first adjustable cuff assembly 250a and second adjustable cuff assembly 250b, also as previously described. In addition to lateral boarder 290, brace 200 comprises medial padded boarder 295 coupled or otherwise fixedly attached to a medial side of fabric portion 230, similar to, but in an opposite orientation from, lateral boarder 290. In some embodiments, brace 200 and/or medial boarder 295 may further include limited or “frameless” plantar surface(s). Lateral boarder 290 and medial boarder 295 provide additional padding and/or bump protection to the lower leg of the wearer of brace 200.

As stated above, all embodiments of brace 200 also, or alternatively, contemplate the use of ratcheting-type cuff assemblies 190. All embodiments of brace 200 also, or alternatively, contemplate securing the first midfoot strap 242a and fastener 244a to a same location on a side of brace 200, for example utilizing a single fastener, and/or securing the second midfoot strap 242b and fastener 244b to a same location on an opposite side of brace 200. Specifically, in contrast to several other embodiments, where fastener 244a and midfoot strap 242a are coupled to fabric portion 230 and/or upright assembly 210 at different locations, in the embodiments shown in at least FIGS. 57-66, both first fastener 244a and a first end of first midfoot strap 242a may be coupled to a lateral side or bottom of fabric portion 230 at a same position, for example, utilizing a single rivet, or rivet and washer combination, 245a (see, e.g., FIG. 58) that is inferior to or distal of the ankle of the wearer while properly disposed in brace 200, or to a similar portion of upright assembly 210 disposed therein. Such embodiments consolidate the fastening of fastener 244a and strap 242a to a single location and fastening feature, for example, similar to that shown in FIG. 58, where strap 142a and the fabric chafe holding the D-ring or plastic chafe, may be sewn together into a single seam and/or fabric feature 144a, through which fastener (e.g., rivet, rivet/washer) 145a passes and secures to fabric portion 130 and/or upright assembly 110 therein. As with such application to brace 100, application to brace 200 also reduces part count and ensures the securing force of the fastener 244a and strap 242a arrangement is directed toward or away from a same location on brace 200 that is inferior to or distal of the ankle of the wearer (e.g., single rivet, or rivet and washer combination, 245a). This advantageously improves consistency and effectiveness of the tension in strap 242a at securing the ankle of the user without the strap bunching, gapping and/or over-tensioning when the user's foot dorsiflexes and/or plantarflexes within brace 200.

Similarly, on the opposite side of brace 200, second fastener 244b and a first end of second midfoot strap 242b may be coupled to a medial side or bottom of fabric portion 230 at a same position, for example, utilizing a single rivet, or rivet and washer combination, 245b (see, e.g., FIG. 57) that is inferior to or distal of the ankle of the wearer, and/or to a similar portion of upright assembly 210 disposed therein. For example, as shown in FIG. 57, strap 242b and the fabric chafe holding the D-ring or plastic chafe, may be sewn together into a single seam and/or fabric feature, through which fastener (e.g., rivet, rivet/washer) 245b passes and secures to fabric portion 230 and/or upright assembly 210 therein. Such embodiments consolidate the fastening of fastener 244b and strap 242b to a single location and fastening feature. This reduces part count and ensures the securing force of the fastener 244b and strap 242b arrangement is directed toward or away from a same location on brace 200 that is inferior to or distal of the ankle of the wearer (e.g., single rivet, or rivet and washer combination, 245b). This advantageously improves effectiveness of maintaining a consistent tension in strap 242b so as to secure the ankle of the user without the strap bunching, gapping and/or over-tensioning when the user's foot dorsiflexes and/or plantarflexes within brace 200.

### Methods of Use

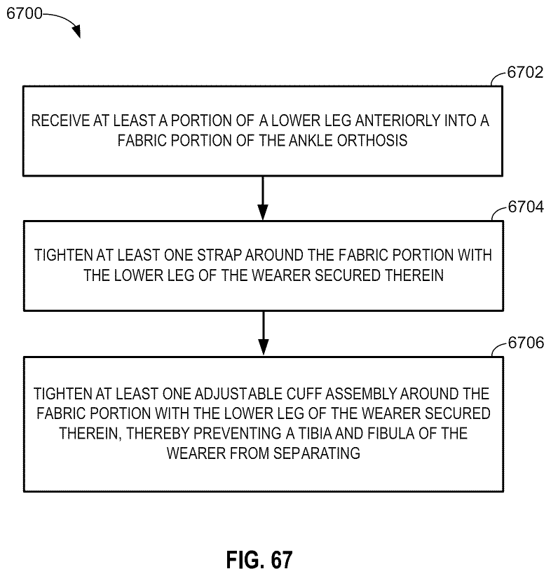

Discussion now turns to one or more methods of utilizing an orthosis (e.g., an ankle brace) configured for attachment to a joint of a subject as described anywhere in this disclosure. For example, FIG. 67 illustrates a flowchart 6700 related to such a method.

Block 6702 includes receiving at least a portion of a lower leg anteriorly into a fabric portion of the orthosis. For example, a wearer or treatment professional may insert a portion of the lower leg of the wearer into an anterior of fabric portion 130 of brace 100, through anterior closure flaps 135, or into fabric portion 230 of brace 200, through similar anterior closure flaps. Such anterior closure flaps may then be secured around the lower leg of the wearer.

Block 6704 includes tightening at least one strap around the fabric portion with the lower leg of the wearer secured therein. For example, the wearer or treatment professional may tighten only midfoot strap 142 around fabric portion 130 of brace 100 while the lower leg of the wearer is secured therein, as described anywhere in this disclosure. As another example, the wearer or treatment professional may tighten midfoot straps 142a and 142b around fabric portion 130 of brace 100 while the lower leg of the wearer is secured therein, as described anywhere in this disclosure. As yet another example, the wearer or treatment professional may tighten midfoot strap 242 and forefoot strap 243 around fabric portion 230 of brace 200 while the lower leg of the wearer is secured therein, as described anywhere in this disclosure. As yet another example, the wearer or treatment professional may tighten first and second midfoot straps 242a, 242b around fabric portion 230 of brace 200 while the lower leg of the wearer is secured therein, as described anywhere in this disclosure.

Block 6706 includes tightening at least one adjustable cuff assembly around the fabric portion with the lower leg of the wearer secured therein, thereby preventing a tibia and fibula of the wearer from separating. For example, the wearer or treatment professional may tighten any adjustable cuff assembly 150, 160, 170, 180, or 190 (e.g., first cuff assembly 190a and/or second cuff assembly 109b) superior or proximal of the ankle of the wearer around fabric portion 130 of brace 100 with the lower leg of the wearer secured therein, as described anywhere in this disclosure. As another example, the wearer or treatment professional may tighten first and second adjustable cuff assemblies 250a, 250b (which may be substantially identical to adjustable cuff assemblies 150, 160, 170 or 180 except as described above regarding sizing around the calf compared to sizing just above the ankle joint) around fabric portion 230 of brace 200 with the lower leg of the wearer secured therein, as described anywhere in this disclosure. As stated above, in yet another example, the wearer or treatment professional may tighten first and second adjustable cuff assemblies that are substantially identical to adjustable cuff assemblies 190a, 190b (except as described above regarding sizing around the calf compared to sizing just above the ankle joint) around fabric portion 230 of brace 200 with the lower leg of the wearer secured therein, as described anywhere in this disclosure.

Such methods of use may alternatively or additionally include any one or more steps for donning, adjusting, tightening, loosening, or otherwise manipulating any aspect of any brace described herein.

### Methods of Manufacture

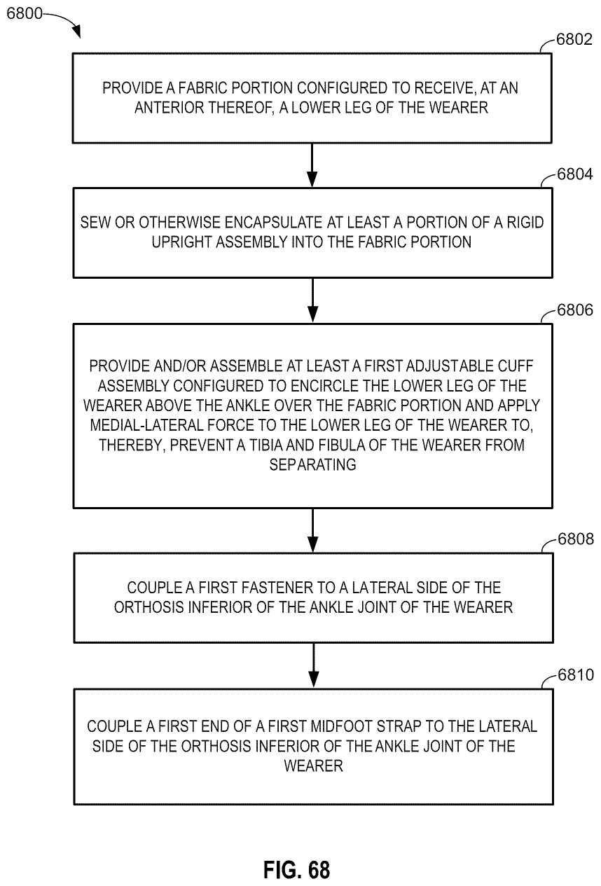

Discussion now turns to one or more methods of manufacturing an orthosis (e.g., an ankle brace) configured for attachment to a joint of a subject as described anywhere in this disclosure. FIG. 68 illustrates a flowchart 6800 of a method of manufacturing an orthosis configured for attachment to a joint of a subject, in accordance with some embodiments. Flowchart 6800 may apply to the manufacture of any brace described in this disclosure.

Block 6802 includes providing a fabric portion configured to receive, at an anterior thereof, a lower leg of the wearer. For example, where brace 100 is manufactured, fabric portion 130 may be provided and/or assembled to be as described anywhere in this disclosure; and where brace 200 is manufactured, fabric portion 230 may be provided and/or assembled to be as described anywhere in this disclosure.

Block 6804 includes sewing or otherwise encapsulating at least a portion of a rigid upright assembly into the fabric portion. For example, where brace 100 is manufactured, at least a portion of rigid upright assembly 110 may be sewn or otherwise encapsulated into fabric portion 130 as described anywhere in this disclosure (e.g., FIG. 5); and where brace 200 is manufactured, at least a portion of rigid upright assembly 210 may be sewn or otherwise encapsulated into fabric portion 130 as described anywhere in this disclosure (e.g., FIG. 37 or FIGS. 38-42).

Block 6806 includes providing and/or assembling at least a first adjustable cuff assembly configured to encircle the lower leg of the wearer above the ankle over the fabric portion and apply medial-lateral force to the lower leg of the wearer to, thereby, prevent a tibia and fibula of the wearer from separating. For example, where brace 100 is manufactured, adjustable cuff assembly 150, 160, 170, 180 or 190 may be provided and/or assembled to be as described anywhere in this disclosure; and where some embodiments of brace 200 are manufactured, first adjustable cuff assembly 250a and second adjustable cuff assembly 250b, which may each be substantially the same as any of assemblies 150, 160, 170 or 180, may be provided and/or assembled to be as described anywhere in this disclosure. In the alternative, where some other embodiments of brace 200 are manufactured, first adjustable cuff assembly and second adjustable cuff assembly may each be substantially the same as assemblies 190a, 190b, as described anywhere in this disclosure.

Block 6808 includes coupling a first fastener to a lateral side of the orthosis inferior of the ankle joint of the wearer. For example, first fastener 144, 144a may be coupled to a lateral side of brace 100 inferior of the ankle joint of the wearer. Where a brace 200 is being manufactured, first fastener 244, 244a may be coupled to a lateral side of brace 200 inferior of the ankle joint of the wearer. As described above, in some embodiments, this fastener may comprise, for example, a rivet and ring arrangement, such as a d-ring, plastic chafe and/or fabric chafe.

Block 6810 includes coupling a first end of a first midfoot strap to the lateral side or a lateral bottom of the orthosis inferior of the ankle joint of the wearer. For example, a first end of midfoot strap 142, 142a may be coupled to the lateral side or a lateral bottom of brace 100 inferior of the ankle joint of the wearer. In alternative example, a first end of midfoot strap 242, 242a may be coupled to the lateral side or a lateral bottom of brace 100 inferior of the ankle joint of the wearer.

In some embodiments, blocks 6808 and 6810 may be combined into a single step. For example, as described in connection with embodiments of both brace 100 and brace 200, where the midfoot strap (142, 142a, 242a) and corresponding fastener (144, 144a, 244b) are coupled to a same location on the orthosis, both the first fastener and the first midfoot strap may be coupled to the same location on the lateral side of brace 100, 200, e.g., via a single rivet, or rivet and washer combination 145a, 245a (see, e.g., FIG. 58).

In some embodiments, such a flowchart or related method of manufacture may additionally include coupling a second fastener to the lateral side of the orthosis inferior of the ankle joint of the wearer and superior or proximal of the first fastener; and coupling a first end of a forefoot strap to the lateral side of the orthosis inferior of the ankle joint of the wearer and inferior of the first end of the first midfoot strap. For example, where some embodiments of brace 200 are manufactured, second fastener 245 may be coupled to the lateral side of brace 200 inferior of the ankle joint of the wearer and superior or proximal of first fastener 244, and a first end of forefoot strap 243 may be coupled to the lateral side of brace 200 inferior of the ankle joint of the wearer and inferior of the first end of midfoot strap 242.

In some other embodiments, such a flowchart or related method of manufacture may additionally include coupling a second fastener to a medial side of the orthosis inferior of the ankle joint of the wearer; and coupling a first end of a second midfoot strap to the medial side or a medial bottom of the orthosis inferior of the ankle joint of the wearer. For example, where some embodiments of brace 100 are manufactured, second fastener 144b may be coupled to a medal side of brace 100 inferior of the ankle joint of the wearer, and a first end of second midfoot strap 142b may be coupled to the medial side or a medial bottom of brace 100 inferior of the ankle joint of the wearer. As described above, in some embodiments, this fastener may comprise, for example, a rivet and ring arrangement, such as a d-ring, plastic chafe and/or fabric chafe.

Where embodiments of brace 200 are manufactured, second fastener 244b may be coupled to a medial side of brace 200 inferior of the ankle joint of the wearer, and a first end of second midfoot strap 242b may be coupled to the medial side or a medial bottom of brace 200 inferior of the ankle joint of the wearer.

Moreover, while several taller ankle braces 200, described herein, are illustrated as having first and second straps 242a, 242b and first and second fasteners 244a, 244b attached at different positions on brace 200, the present disclosure is not so limited, and the present disclosure also contemplates tall braces 200 as otherwise described anywhere herein, alternatively having first strap 242a and corresponding fastener 244a being coupled to the same position on fabric portion 230 and/or upright assembly 210 utilizing for example a single rivet, or rivet and washer combination, 245a (see, e.g., FIG. 58) that is inferior to or distal of the ankle of the wearer, or to a similar portion of upright assembly 210 disposed therein. Similarly, second strap 242b and corresponding fastener 244b may be coupled to the same position on fabric portion 230 and/or upright assembly 210 utilizing for example a single rivet, or rivet and washer combination, 245b (see, e.g., FIG. 57) that is inferior to or distal of the ankle of the wearer, or to a similar portion of upright assembly 210 disposed therein.

Such methods of manufacture may alternatively or additionally include any one or more steps for providing, assembling or creating any aspect of any brace described herein.

Although the present disclosure has been described in terms of certain preferred features, other features of the disclosure including variations in dimensions, configuration and materials will be apparent to those of skill in the art in view of the disclosure herein. In addition, all features detailed in connection with any embodiment herein can be readily adapted for use in any other embodiment herein. The use of different terms or reference numerals for similar features in different embodiments does not imply differences other than those which may be expressly set forth. Accordingly, the present disclosure is intended to be described solely by reference to the appended claims, and not limited to the preferred embodiments disclosed herein.

What is claimed is:

1. An ankle orthosis configured for attachment around an ankle of a wearer, the orthosis comprising:
a fabric portion comprising closure flaps, the fabric portion configured to receive a lower leg of the wearer through the closable flaps;
a rigid upright assembly, wherein at least a portion of the rigid upright assembly is disposed within the fabric portion;
at least one adjustable cuff assembly configured to encircle the lower leg of the wearer above the ankle over the fabric portion and apply medial-lateral force to the lower leg of the wearer, thereby, preventing a tibia and fibula of the wearer from separating;
a first fastener coupled to a lateral side of the orthosis at a position inferior of the ankle joint of the wearer; and
a first midfoot strap configured to wrap at least partly around the midfoot of the wearer over the fabric portion, the first midfoot strap comprising:
a first end coupled to the same position on the lateral side of the orthosis as the first fastener, and
a second end configured to couple to the first fastener.

2. The orthosis of claim 1, wherein the fabric portion comprises at least one of:
a first portion having a desired amount of stretchability and comprising the closure flaps, which are disposed at an anterior of the fabric portion, wherein securing the closure flaps around the lower leg of the wearer forms a foot hole at the distal end of the fabric portion and a lower leg hole at the proximal end of the fabric portion;
a heel hole disposed in a posterior portion of the fabric portion, between the foot hole and the lower leg hole;
a second portion, having a lower degree of stretchability than the first portion, disposed on medial and lateral sides of the fabric portion and configured to encase at least a portion of the upright assembly; and
a posterior spine configured to be disposed along an Achilles tendon of the wearer and comprising a polypropylene webbing or seam tape.

3. The orthosis of claim 1, wherein the rigid upright assembly comprises:
an underfoot portion that bends to form lateral and medial lower uprights extending substantially perpendicular to the underfoot portion;
a lateral upper upright having a proximal end pivotally coupled to a distal end of the lateral lower upright by a lateral joint; and
a medial upper upright having a proximal end pivotally coupled to a distal end of the medial lower upright by a medial joint, the medal and lateral upper uprights each extending proximally to approximately half way between the ankle and the calf of the wearer when the orthosis is donned by the wearer.

4. The orthosis of claim 1, wherein the rigid upright assembly comprises:
an underfoot portion that bends to form lateral and medial lower uprights extending substantially perpendicular to the underfoot portion;
a medial upper upright having a distal end coupled to a proximal end of the medial lower upright by a medial joint, wherein a proximal end of the medial upper upright extends to the calf when the orthosis is donned by the wearer; and
wherein the lateral lower upright extends, unjointed, to the calf when the orthosis is donned by the wearer, thereby providing a diagnosis-specific torsional affect opposing or resisting external rotation of the ankle of the wearer.

5. The orthosis of claim 1, wherein the rigid upright assembly comprises:
an underfoot portion that bends to form lateral and medial lower uprights extending substantially perpendicular to the underfoot portion;
a medial upper upright having a distal end coupled to a proximal end of the medial lower upright by a medial joint, wherein a proximal end of the medial upper upright extends to the calf when the orthosis is donned by the wearer; and
a lateral upper upright having a distal end coupled to a proximal end of the lateral lower upright by a pivot structure comprising a plurality of fingers, each finger comprising:
a first portion coupled to and extending substantially straight away from the lower lateral upright and substantially, and
a second portion extending from the first portion and having an oblong curvature that ultimately couples with the lateral upper upright,
wherein, when the lateral upper upright is bent with respect to the lateral lower upright, the second portion of each of the fingers bends but remains in a common plane.

6. The orthosis of claim 1, wherein each of the at least one adjustable cuff assembly comprises:
a posterior cuff portion configured to be disposed against posterior portions of the lower leg of the user above the ankle;
a first anterior cuff portion pivotally coupled to a first end of the posterior cuff portion;
a second anterior cuff portion slidingly coupled to a second end of the posterior cuff portion; and
a third anterior cuff portion pivotally coupled to the second anterior cuff portion, wherein the first, second and third cuff portions are configured to be disposed against respective portions of a side or anterior of the lower leg of the user above the ankle.

7. The orthosis of claim 6, wherein the posterior cuff portion comprises a post and the second anterior cuff portion comprises a slot configured to slidingly receive the post.

8. The orthosis of claim 6, wherein the slot extends substantially parallel to a length of circumferential extension of posterior cuff portion about the lower leg of the wearer.

9. The orthosis of claim 6, wherein the slot is disposed toward an opposite end of the second anterior cuff portion from an end that is pivotally attached to the third anterior cuff portion.

10. The orthosis of claim 6, wherein at least one surface of the first anterior cuff portion comprises a plurality of ratcheting teeth configured to be received by a complementary locking feature.

11. The orthosis of claim 6, wherein the third anterior cuff portion comprises an aperture configured to receive at least a terminal end of the first anterior cuff therethrough.

12. The orthosis of claim 6, wherein the at least one adjustable cuff assembly comprises:
a first adjustable cuff assembly configured to be disposed adjacent a proximal edge of the fabric portion and proximal of the ankle of the wearer; and
a second adjustable cuff assembly configured to be disposed proximal of the ankle of the wearer and distal of the first adjustable cuff assembly.

13. The orthosis of claim 1, wherein the fabric portion is configured to encircle the lower leg of the wearer from a forefoot of the wearer to approximately half way between the ankle and a calf of the wearer.

14. The orthosis of claim 1, wherein the fabric portion is configured to encircle the lower leg of the wearer from a forefoot of the wearer to a calf of the wearer.

15. The orthosis of claim 1, wherein, when properly secured around the lower leg of the wearer, the first midfoot strap extends from the first end:
under the foot of the wearer,
up a medial side of the midfoot of the wearer,
over a top of the foot of the wearer distal of the ankle,
through a slot or around a bar of the first fastener, and
doubling back and securing to itself inferior of the ankle, thereby, providing adjustable support at the midfoot of the wearer.

16. The orthosis of claim 1, wherein the second end of the first midfoot strap comprises at least one of hook and look fasteners and complementary snap fasteners.

17. The orthosis of claim 1, further comprising:
a second fastener coupled to a medial side of the orthosis at a position inferior of the ankle joint of the wearer; and
a second midfoot strap configured to wrap at least partly around the midfoot of the wearer over the fabric portion, the second midfoot strap comprising:
a first end coupled to the same position on the medial side of the orthosis as the second fastener, and
a second end configured to couple to the first fastener.

18. The orthosis of claim 17, wherein, when properly secured around the lower leg of the wearer, the second midfoot strap extends from the first end:
under the foot of the wearer,
up a lateral side of the midfoot of the wearer,
over a top of the foot of the wearer distal of the ankle,
through a slot or around a bar of the second fastener, and
doubling back and securing to itself inferior of the ankle, thereby, providing adjustable support at the midfoot of the wearer.

19. The orthosis of claim 17, wherein, for at least one of the first and second midfoot straps, at least the portion of the strap configured to be disposed against an underside of the arch of the foot of the wearer has a first width and tapers to a second width that is narrower than the first width as the strap extends toward the second end.

20. The ankle orthosis of claim 1, further comprising at least one of:
a lateral padded boarder coupled to and extending substantially along an entire lateral extent of the fabric portion; and
a medial padded boarder coupled to and extending substantially along an entire medial extent of the fabric portion.

21. A method of utilizing an ankle orthosis configured for attachment to an ankle of a wearer, the method including:
receiving at least a portion of a lower leg anteriorly into a fabric portion of the ankle orthosis;
tightening at least one strap around the fabric portion with the lower leg of the wearer secured therein; and
tightening at least one adjustable cuff assembly around the fabric portion with the lower leg of the wearer secured therein, thereby preventing a tibia and fibula of the wearer from separating.

22. A method of manufacturing an ankle orthosis configured for attachment around an ankle of a wearer, the method comprising:
providing a fabric portion comprising closure flaps, the fabric portion configured to receive a lower leg of the wearer through the closable flaps;
sewing or otherwise encapsulating at least a portion of a rigid upright assembly into the fabric portion;
providing and/or assembling at least a first adjustable cuff assembly configured to encircle the lower leg of the wearer above the ankle over the fabric portion and apply medial-lateral force to the lower leg of the wearer to, thereby, prevent a tibia and fibula of the wearer from separating;
coupling a first fastener to a lateral side of the orthosis at a position inferior of the ankle joint of the wearer; and
coupling a first end of a first midfoot strap to the same position on the lateral side of the orthosis as the first fastener.

23. The method of claim 22, further comprising:
coupling a second fastener to a lateral side of the orthosis at a position inferior of the ankle joint of the wearer; and
coupling a first end of a second midfoot strap to the same position on the medial side of the orthosis as the second fastener.
